(19) United States
(12) Patent Application Publication
(10) Pub. No.: US 2016/0045620 A1
(43) Pub. Date: Feb. 18, 2016

(54) USE OF NANOMETER-SIZED PRODRUGS OF NSAIDs TO TREAT TRAUMATIC BRAIN INJURY

(71) Applicant: Cedars-Sinai Medical Center, Los Angeles, CA (US)

(72) Inventors: John S. Yu, Los Angeles, CA (US); Bong Seop Lee, Torrance, CA (US)

(73) Assignee: Cedars-Sinai Medical Center, Los Angeles, CA (US)

(21) Appl. No.: 14/782,677

(22) PCT Filed: Apr. 18, 2014
(86) PCT No.: PCT/US14/34691

§ 371 Date: Oct. 6, 2015

Related U.S. Application Data
(60) Provisional application No. 61/814,141, filed on Apr. 19, 2013.

(51) Int. Cl. A61K 49/00; A61K 47/48; A61K 31/355; A61K 31/216

(52) U.S. Cl. CPC A61K 49/0052; A61K 31/216; A61K 47/48107; A61K 31/355; A61K 47/48023

(57) ABSTRACT

The present invention describes methods of delivering derivatives of non-steroidal anti-inflammatory drugs (NSAIDs) and nanospheres thereof as well as therapeutic agents to the injured brain tissue in subjects with traumatic brain injury. The invention also provides methods of treating traumatic brain injury and related conditions. The invention further provides methods of diagnosing traumatic brain injury.

### CROSS-REFERENCE TO RELATED APPLICATIONS

This application claims the benefit under 35 U.S.C. §119(e) of U.S. Provisional Application No. 61/814,141 filed Apr. 19, 2013, the content of which is incorporated herein by reference in its entirety.

### FIELD OF INVENTION

This invention relates to uses of derivatives of non-steroidal anti-inflammatory drugs (NSAIDs) and nanospheres thereof to treat traumatic brain injury and related conditions.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### Traumatic Brain Injury (TBI):

In industrialized countries, traumatic brain injury (TBI) is the leading cause of death in those under the age of 45 [1-3] and traumatic injuries account for a greater number of potential years of life lost than all other causes of death [4]. According to most recent CDC estimates (2004-2006), there are 1.7 million new cases of TBI annually, with 52,000 deaths, 275,000 hospitalizations, and 1.4 million people treated in emergency departments each year [5].

For both patients and society at large, traumatic brain injury carries a large cost burden estimated for the United States to be $60-100 billion per year due to resulting healthcare costs and lost productivity [6,7]. For survivors, the impact of TBI on quality of life is significant, as most suffer some degree of cognitive impairment that may include memory or motor deficits, psychological disorders, sleep disturbances, or seizures, with an increased risk for developing neurodegenerative diseases or other encephalopathies later in life [8,9]. Currently, an estimated 5.3 million people live in the US with a permanent TBI related disability [5].

Decades of clinical and basic science trials have attempted to improve outcomes of traumatic brain injury using a wide variety of novel treatment strategies. The most recent trials have investigated drugs such as calcium channel inhibitors [10-12], dexanabinol [13,14], minocycline [15,16] and magnesium [17,18]. Unfortunately, no interventions have been successful enough in practice to be implemented as standard of care [19-21]. Accordingly, there is a need for an effective treatment for traumatic brain injury.

Among the animal models available for traumatic brain injury, the controlled cortical impact (CCI) method represents a refined and highly reproducible means of producing several gradations of injury [22]. CCI can be conducted as either a closed or open injury with or without burr hole exposure of the brain. The CCI model, first described in 1988 by Lighthall et al., allows for the control of multiple parameters of injury, including the velocity, duration, penetration depth, and contact area of the impact [23]. Other commonly used models include fluid percussion injury, first described in 1965 by Lindgren and Rinder [24], and weight drop described by Feeney et al in 1981 [25]. Very recently, mouse models for blast injury have been developed for the purpose of reproducing the common battlefield injury, and investigating the distinct pathologies associated with this mechanism of injury [26,27].

CCI recapitulates the characteristics of human traumatic brain injury such as edema, hemorrhage, contusion, altered cerebral metabolism and inflammation [28,29]. Clinical traumatic brain injury is broadly categorized as blunt or penetrating. The majority of TBIs are blunt injuries, in which there is a direct impact to the skull without penetration of the intracranial space. The leading causes of blunt injury are falls for the age groups 0-14 and those over 35, while motor vehicle accidents are the leading cause for those between the ages of 14 and 35 [5]. Penetrating injuries result from mechanisms of injury such as gunshot wounds and shrapnel, and differ substantially in management and prognosis. Traumatic brain injury has gained significant attention due to its prevalence in recent military conflicts, where an estimated 28% of combat casualties sustain TBI [30,31].

### SUMMARY OF THE INVENTION

The following embodiments and aspects thereof are described and illustrated in conjunction with compositions and methods which are meant to be exemplary and illustrative, not limiting in scope.

The present disclosure provides for methods of using nanospheres formed from the molecules as described herein. In one aspect, the disclosure provides a method of delivering a nanosphere to a traumatic brain injury (TBI) tissue in a subject, comprising administering a therapeutically effective amount of a nanosphere to the subject, wherein the nanosphere comprises a compound selected from Formula B-I, Formula B-II, Formula B-III, and any combinations thereof.

In various embodiments, the method can be used to deliver a NSAID to a TBI tissue in a subject, the method comprising administering a therapeutically effective amount of a nanosphere to the subject, wherein the nanosphere comprises a compound selected from Formula B-I, Formula B-II, Formula B-III, and any combinations thereof.

NSAIDs have properties that are beneficial to TBI patients. Thus, the method of delivering NSAID to TBI tissue may be used for treating TBI in a subject. Accordingly, in some embodiments, the method of delivering the nanosphere to the TBI tissue can treat TBI. Since the method of delivering the nanosphere to TBI tissue can treat TBI, the disclosure also provides a method of treating TBI in a subject in need thereof, comprising: providing a nanosphere comprising a compound of Formula B-I, Formula B-II, or Formula B-III or any combinations thereof; and administering a therapeutically effective amount of the nanosphere to the subject to treat the TBI.

In another aspect, the disclosure provides a method of detecting or diagnosing TBI in a subject in need thereof comprising: providing a nanosphere comprising a compound of Formula B-I, Formula B-II, or Formula B-III or any combinations thereof, and an imaging agent; administering an effective amount of the nanosphere to the subject; and imaging the subject to detect or diagnose the TBI. In some embodiments, the imaging agent can be conjugated to a hydrophilic spacer, a hydrophobic spacer, an amphiphilic spacer, or an amphiphilic spacer.

The present disclosure also provides a nanosphere comprising a compound formula B-I, B-II, B-III, or any combinations thereof. These nanospheres can be those that are delivered to TBI tissue. Thus, the present disclosure also provides a compound of formula B-I, B-II, or B-III.

In the compounds of formula B-I:

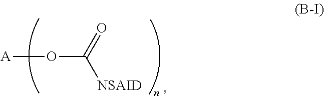

variable A can be selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; n can be an integer of at least two; and NSAID is a nonsteroidal anti-inflammatory drug.

In compounds of formula B-II:

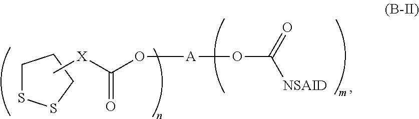

variable A can be selected from the group consisting of a substituted, unsubstituted, branched or unbranched chain of carbon atoms and can optionally contain a heteroatom; A can be selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; n can be an integer of at least one; m can be an integer of at least one; and NSAID is a nonsteroidal anti-inflammatory drug.

In some embodiment, the dithiolane moiety in molecules of formula B-II can be an α-lipoic acid (“ALA”). Accordingly, a molecule of formula B-II can be of formula B-III:

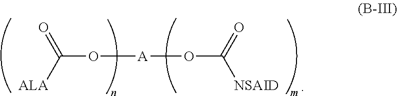

In compounds of formula B-I, B-II or B-III, NSAID can be selected from the group consisting of aspirin, ibuprofen, flurbiprofen, ketoprofen, fenoprofen, fenbufen, naproxen, indomethacin, diclofenac, ketorolac, tolmetin, flufenamic acid, mefenamic acid, tolfenamic acid, meclofenamic acid, niflumic acid, sulindac, sulindac sulfide and any combinations thereof.

In various embodiments, the nanosphere further comprises a compound having Formula A-IV or Formula A-V. Thus, the present disclosure also provides a compound of formula A-IV or A-V. In compounds of formula A-IV:

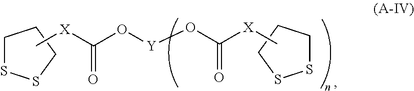

X can be selected from the group consisting of a substituted, unsubstituted, branched or unbranched chain of carbon atoms and can optionally contain a heteroatom; Y can be selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; and n can be an integer of at least one.

In some embodiment, at least one dithiolane moiety in a molecule of formula A-IV can be an α-lipoic acid, i.e. (R)-5-(1,2-dithiolan-3-yl)pentanoic acid. Thus, in some embodiments, a molecule of formula A-IV can be of formula A-V:

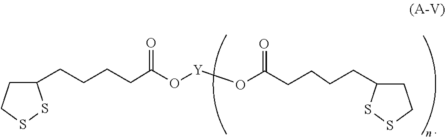

In various embodiments, the nanosphere further comprises an antioxidant. In some embodiments, the antioxidant can be conjugated to a hydrophilic spacer, a hydrophobic spacer, an amphiphilic spacer, or an amphiphilic polymer. In some embodiments, the antioxidant is tocopherol or a derivative or analogue thereof. In some embodiments, the antioxidant can be glutathione, a hydrophobic derivative of glutathione, N-acetyl cysteine, or a hydrophobic derivative of N-acetyl glutathione.

In various embodiments, the nanosphere further comprises a therapeutic agent. In certain embodiments, the delivery of the nanosphere further comprising to the therapeutic agent delivers the therapeutic agent to the TBI tissue. In various embodiments, the delivery of the therapeutic agent to the TBI tissue treats TBI.

In some embodiments, the therapeutic agent is conjugated to a hydrophilic spacer, a hydrophobic spacer, an amphiphilic spacer, or an amphiphilic polymer. In some embodiments, the therapeutic agent is selected from the group consisting of: a statin, nonsteroidal anti-inflammatory drug (NSAID), erythropoietin, peptide, antisense nucleic acid, DNA, RNA, protein, and combinations thereof.

In various embodiments, the nanosphere further comprises an imaging agent. In some embodiments, the imaging agent is conjugated to a hydrophilic spacer, a hydrophobic spacer, an amphiphilic spacer, or an amphiphilic polymer. In some embodiments, the imaging agent is selected from the group consisting of fluorescent molecules, radioisotopes, nucleotide chromophores, enzymes, substrates, chemiluminescent moieties, bioluminescent moieties, and any combinations thereof. In some embodiments, the imaging agent is a fluorophore.

In various embodiments, the nanosphere can further comprise an amphiphilic spacer. In some embodiments, the amphiphilic spacer comprises a chemically active group selected from the group consisting of thiol, amine, carboxylic acid, carboxylic acid NHS ester, maleimide, hydrazine, ketone, aledehyde, and combinations thereof. In some embodiments, the amphiphilic spacer is an alkylthiol or an alkylamine. One exemplary alkylthio is 1-octadecanethiol.

In various embodiments, the nanosphere can further comprise a polymer. In various embodiments, the polymer can be selected from the group consisting of a hydrophobic polymer, amphiphilic polymer, and hydrophobically modified hydrophilic polymer. In other embodiments, the polymer can be selected from the group consisting of a polyanhydride, polyester, polyorthoester, polyesteramide, polyacetal, polyketal, polycarbonate, polyphosphoester, polyphosphazene, polyvinylpyrrolidone, polydioxanone, poly(malic acid), poly(amino acid), polymer of N-2-(hydroxypropyl)methacrylamide (HPMA), polymer of N-isopropyl acrylamide (NIPAAm), polyglycolide, polylactide, copolymer of glycolide and lactide (e.g., poly(lactide-co-glycolide), and combinations thereof. In some embodiments, the polymer is poly(lactide-co-glycolide) (PLGA).

In various embodiments, the polymer can contain a side group selected from the group consisting of a hydrophobic molecule, hydrophilic molecule, and amphiphilic molecule. In various embodiments, the side group can be a therapeutic or diagnostic agent. In other embodiments, the therapeutic agent can be selected from the group consisting of a peptide, antisense nucleic acid, and protein. In additional embodiments, the polymer can contain a hydrophobic side groups selected from the group consisting of an aromatic group, amino acid alkyl ester, and aliphatic group.

The present disclosure also provides for a method of treating a disease condition in a subject in need thereof, comprising: providing a therapeutically effective quantity of a nanosphere of described herein, and administering the therapeutically effective quantity to the subject.

The disclosure also provides for a method of delivering a therapeutic agent, comprising: providing a composition comprising the therapeutic agent and a nanosphere of the described herein; and administering the composition to the subject.

The disclosure also provides for a composition comprising: a nanosphere comprising a molecule of formula B-I, B-II or B-III; and a nanosphere comprising a molecule of formula A-IV or A-V.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts the nanoprodrug preparation and characterization. This chemical schematic shows the molecular structures of the individual ibuprofen molecule, the Ibu₂TEG complex consisting of two ibuprofen molecules jointed by a tetraethylene glycol (TEG) spacer, the antioxidant α-tocopherol, and the hydrophobic 1-octadecanethiol which is joined to Cy5.5 after emulsification. The final product is represented on the right hand side of the schematic.

FIG. 2 illustrates the comparison of the accumulation for IV and IP administration. The injection of nanoprodrug either IV or IP results in similar accumulation in animals with TBI, while normal animals given nanoprodrug and TBI animals do not show any background fluorescence. Brains are oriented with the rostral portion toward the top of the image. Control: no TBI. Nanoprodrug: fluorescently labeled NSAID nanoprodrug. PBS: phosphate buffered saline

FIG. 3 depicts the drug accumulation in the area of injury. Accumulation of the drug in the left parietal area is visualized (a) using fluorescent imaging in the top panels and (b) by traditional photography and hematoxylin and eosin staining in the lower panels.

FIG. 4 depicts the disorganized vascular structures at the region of nanoprodrug uptake. Representative images from two brains showing nanoprodrug uptake on the left column and CD31 staining of vascular endothelial cells on the right. Outside of the TBI region, vascular structures exist in normal tubular arrangements, but these are disorganized within the region of injury. The nuclei are stained with DAPI are displayed in blue. Scale bar, 50 μm. NB: Normal brain (injury periphery); TBI: Traumatic brain injury (focal insult).

FIGS. 5a-c depict behavioral testing of motor function using Open Field Test and Rotorod. (a) The number of ambulatory movements over the course of one hour in the Open Field Test (OFT) was reduced for the mice in the IV group. (b) The number of rearing movements in the OFT was not significantly different between groups. (c) Rotorod performance demonstrates that all mice were able to balance on a rotating rod for similar amounts of time. Statistical comparison was performed using a two tailed Student's t-test, with a level of p=0.05 considered significant.

FIG. 6 depicts behavioral testing of memory function using the Barnes Maze. The Barnes Maze was conducted for five days of training followed by traumatic brain injury (TBI). One day after traumatic brain injury (Day 7 on graph), mice did not demonstrate any significant differences in time to find the escape box. Statistical comparison was performed using a two tailed Student's t-test, with a level of p=0.05 considered significant. Day 8 serves as a control in which the location of the box is changed to show that mice are not using other cues such as scent to locate the box.

FIG. 7 depicts the Enhanced Permeability and Retention (EPR) at the site of TBI. This schematic illustrates the difference between healthy brain and injured brain in terms of the structural organization of the blood vessels and how this influences drug delivery. In normal tissue, the blood brain barrier is intact and the nanoprodrug does not penetrate into the tissue. An injured vessel becomes leaky, and the disruption of the blood brain barrier allows for uptake and accumulation of the nanoprodrug particles.

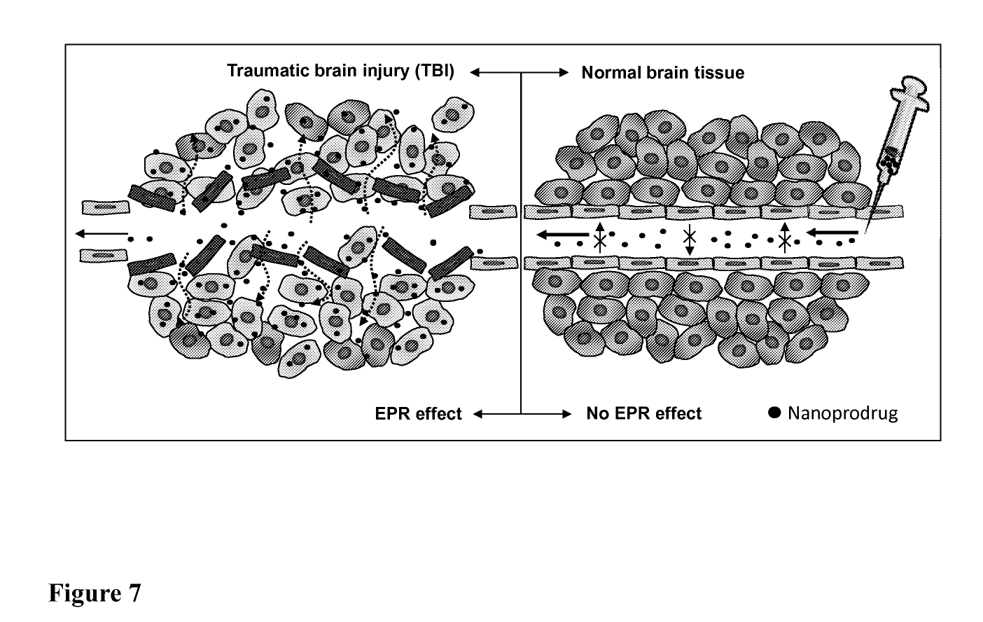

FIG. 8 depicts the COX system regulating blood flow and platelet activity. The COX1 enzyme acts in platelets to activate thromboxane A2, which leads to vasoconstriction and enhanced platelet aggregation. The COX2 enzyme acts in endothelial cells to stimulate vasorelaxation and platelet inhibition.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N. Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Nanosphere comprising NSAID” and “Nanosphere prodrug comprising NSAID” as used herein refer to a nanosphere comprising a NSAID compound. In some embodiments, the NSAID is ibuprofen. In some embodiments, the NSAID is Ibu2TEG. The nanosphere can further comprise a multiple α-lipoic acid-containing hydrophobic compound, α-tocopherol, an additional nonsteroidal anti-inflammatory drug (NSAID) derivative, or combinations thereof.

“Beneficial results” can include, but are in no way limited to, lessening or alleviating the severity of the disease condition, preventing the disease condition from worsening, curing the disease condition and prolonging a patient's life or life expectancy. The disease conditions can relate to or can be modulated by the central nervous system.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein.

In some embodiments, the subject is a mammal. “Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term. Mammals other than humans can be advantageously used as subjects that represent animal models of disorders.

“Nanosphere” as used herein refers to a particle with a size, in at least one dimension, between about 10 nm to about 1000 nm; and can also include a nanoemulsion. It will be understood by one of ordinary skill in the art that particles usually exhibit a distribution of particle sizes around the indicated “size.” Unless otherwise stated, the term “particle size” as used herein refers to the mode of a size distribution of particles, i.e., the value that occurs most frequently in the size distribution. Methods for measuring the particle size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

In some embodiments, the particles can be substantially spherical. What is meant by “substantially spherical” is that the ratio of the lengths of the longest to the shortest perpendicular axis of the particle cross section is less than or equal to about 1.5. Substantially spherical does not require a line of symmetry. Further, the particles can have surface texturing, such as lines or indentations or protuberances that are small in scale when compared to the overall size of the particle and still be substantially spherical. In some embodiments, the ratio of lengths between the longest and shortest axes of the particle is less than or equal to about 1.5, less than or equal to about 1.45, less than or equal to about 1.4, less than or equal to about 1.35, less than or equal to about 1.30, less than or equal to about 1.25, less than or equal to about 1.20, less than or equal to about 1.15 less than or equal to about 1.1. Without wishing to be bound by a theory, surface contact is minimized in particles that are substantially spherical, which minimizes the undesirable agglomeration of the particles upon storage. Many crystals or flakes have flat surfaces that can allow large surface contact areas where agglomeration can occur by ionic or non-ionic interactions. A sphere permits contact over a much smaller area.

The particles can be, e.g., monodispersed or polydispersed and the variation in diameter of the particles of a given dispersion can vary. In some embodiments, the particles have substantially the same particle size. Particles having a broad size distribution where there are both relatively big and small particles allow for the smaller particles to fill in the gaps between the larger particles, thereby creating new contact surfaces. A broad size distribution can result in larger spheres by creating many contact opportunities for binding agglomeration. The particles described herein are within a narrow size distribution, thereby minimizing opportunities for contact agglomeration. What is meant by a “narrow size distribution” is a particle size distribution that has a ratio of the volume diameter of the 90th percentile of the small spherical particles to the volume diameter of the 10th percentile less than or equal to 5. In some embodiments, the volume diameter of the 90th percentile of the small spherical particles to the volume diameter of the 10th percentile is less than or equal to 4.5, less than or equal to 4, less than or equal to 3.5, less than or equal to 3, less than or equal to 2.5, less than or equal to 2, less than or equal to 1.5, less than or equal to 1.45, less than or equal to 1.40, less than or equal to 1.35, less than or equal to 1.3, less than or equal to 1.25, less than or equal to 1.20, less than or equal to 1.15, or less than or equal to 1.1.

Geometric Standard Deviation (GSD) can also be used to indicate the narrow size distribution. GSD calculations involved determining the effective cutoff diameter (ECD) at the cumulative less than percentages of 15.9% and 84.1%. GSD is equal to the square root of the ratio of the ECD less than 84.17% to ECD less than 15.9%. The GSD has a narrow size distribution when GSD<2.5. In some embodiments, GSD is less than 2, less than 1.75, or less than 1.5. In one embodiment, GSD is less than 1.8.

“Nanoprodrug” is used interchangeably with “nanosphere” throughout the application.

“Non-steroidal” as used herein distinguishes the anti-inflammatory drugs from steroids, which have a similar anti-inflammatory action.

“NSAID derivative” as used herein refers to a compound in which at least one NSAID molecule is coupled to a polyol (for example, through esterification), coupled to an amine, or coupled to an aminoalcohol.

“NSAID nanosphere” as used herein refers to a nanosphere comprising molecules of Formula B-I, B-II, B-III, or any combinations thereof.

“Polyol” as used herein refers to a compound that contains at least two free esterifiable hydroxyl groups.

“Therapeutic agent” as used herein refers to any substance used internally or externally as a medicine for the treatment, cure, prevention, slowing down, or lessening of a disease or disorder, even if the treatment, cure, prevention, slowing down, or lessening of the disease or disorder is ultimately unsuccessful. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent can be used which are capable of being released from the subject composition into adjacent tissues or fluids upon administration to a subject. Examples include steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antivirals, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., antisense oligonucleotides that bind to a target nucleic acid sequence (e.g., mRNA sequence)), peptides, proteins, antibodies, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals.

“Therapeutically effective amount” as used herein refers to an amount which is capable of achieving beneficial results in a patient with a condition or a disease condition in which treatment is sought. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein refer to both therapeutic treatment and prophylactic or preventative measures, wherein the object is to prevent, slow down and/or alleviate the disease or disease condition even if the treatment is ultimately unsuccessful. Thus, the terms “treatment” and “treating” encompass therapeutic treatments, wherein the object is to reverse, alleviate, ameliorate, inhibit, slow down or stop the progression or severity of a condition associated with a disease or disorder, e.g. TBI. The term “treating” includes reducing or alleviating at least one adverse effect or symptom of a condition, disease or disorder associated with TBI. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “antioxidant agent” refers to a molecule that decreases, inhibits, prevents, or reduces the oxidation of an oxidizable compound. Without limitations, a compound is considered an antioxidant for purposes of this disclosure if it reduces endogenous oxygen radicals in vitro. As nonlimiting examples, antioxidants scavenge oxygen, superoxide anions, hydrogen peroxide, superoxide radicals, lipooxide radicals, hydroxyl radicals, or bind to reactive metals to prevent oxidation damage to lipids, proteins, nucleic acids, etc. Antioxidants remove free radical intermediates and inhibit other oxidation reactions by being oxidized themselves. Examples of antioxidants include, but are not limited to, hydrophilic antioxidants, lipophilic antioxidants, and mixtures thereof. Non-limiting examples of hydrophilic antioxidants include chelating agents (e.g., metal chelators) such as ethylenediaminetetraacetic acid (EDTA), citrate, ethylene glycol tetraacetic acid (EGTA), 1,2-bis(o-aminophenoxy)ethane-N,N,N′,N′-tetraacetic acid (BAPTA), diethylene triamine pentaacetic acid (DTP A), 2,3-dimercapto-1-propanesulfonic acid (DMPS), dimercaptosuccinic acid (DMSA), cc-lipoic acid, salicylaldehyde isonicotinoyl hydrazone (SIH), hexyl thioethylamine hydrochloride (HTA), desferrioxamine, salts thereof, and mixtures thereof. Additional hydrophilic antioxidants include ascorbic acid, cysteine, N-acetyl cysteine, hydrophobic derivatives of N-acetyl cysteine, glutathione hydrophobic derivative of glutathione, dihydrolipoic acid, 2-mercaptoethane sulfonic acid, 2-mercaptobenzimidazole sulfonic acid, 6-hydroxy-2,5,7,8-tetramethylchroman-2-carboxylic acid, sodium metabisulfite, salts thereof, and mixtures thereof. Non-limiting examples of lipophilic antioxidants include vitamin E isomers such as α-, β-, γ-, and δ-tocopherols and α-, β-, γ-, and δ-tocotrienols; polyphenols such as 2-tert-butyl-4-methyl phenol, 2-tert-butyl-5-methyl phenol, and 2-tert-butyl-6-methyl phenol; butylated hydroxyanisole (BHA) (e.g., 2-teri-butyl-4-hydroxyanisole and 3-tert-butyl-4-hydroxyanisole); butylhydroxytoluene (BHT); quinones, e.g., tert-butylhydroquinone (TBHQ); ascorbyl palmitate; n-propyl gallate; salts thereof; and mixtures thereof.

In some embodiments, the antioxidant agent can be glutathione, hydrophobic derivative of glutathione, N-acetyl cysteine, hydrophobic, or hydrophobic derivatives of N-acetyl cysteine.

As used herein, a hydrophobic derivative of glutathione refers to a glutathione derivative comprising at least one hydrophobic group attached to one of the carboxylate groups or the amine group of glutathione. For example, the hydrophobic group can form an ester or amide with the glutathione.

As used herein, a hydrophobic derivative of N-acetyl-cysteine means a N-acetyl cysteine comprising a hydrophobic group attached to the carboxylate group of the N-acetyl cysteine. In some embodiments, the hydrophobic group can form an ester with the N-acetyl cysteine.

As used herein, the term “hydrophobic group” refers to those groups being immiscible in water. The terms “hydrophobic group” refers to any of the groups hydrogen, alkyl, alkoxy, alkoxyalkyl, aryloxy, cycloalkoxy, alkylthio, alkenyl, alkynyl, cycloalkyl, haloalkyl, alkanoyl, aroyl, aminocarbonyl, aminoalkanoyl or optionally substituted aminoalkanoyl, carbocycloalkyl or optionally substituted carbocycloalkyl, heterocyclo or optionally substituted heterocyclo, heteroaryl or optionally substituted heteroaryl, halo, aryl, aralkyl, (heterocyclo)alkyl, (heteroaryl)alkyl, alkoxycarbonyl, alkylcarbonyloxy, alkoxyalkanoyl, carboxyalkyl, amino or substituted amino, amido or substituted amido, and alkanoylamido having at least some affinity for a hydrocarbon. In some embodiments, suitable hydrophobic groups include normal or branched C₁-C₁₈-alkyl groups, arylalkyl groups and aryl groups. In some embodiments, the hydrophobic group can be a C1-18 alkyl.

As discussed elsewhere in the disclosure, the antioxidant agents can be included in the matrix of the nanoparticles or conjugated with a hydrophilic spacer, a hydrophobic spacer, an amphiphilic spacer, or an amphiphilic polymer in the nanosphere. Without wishing to be bound by a theory, an antioxidant agent conjugated with a hydrophilic spacer, a hydrophobic spacer, an amphiphilic spacer, or an amphiphilic polymer in the nanosphere can be present on the surface of the nanosphere. Accordingly, unmodified glutathione and N-acetyl-cysteine can be present conjugated on the surface of the nanosphere by conjugating with a hydrophilic spacer, a hydrophobic spacer, an amphiphilic spacer, or an amphiphilic polymer in the nanosphere. Hydrophobically modified glutathione and N-acetyl-cysteine derivatives can be included in the matrix of the nanoparticles.

“Antioxidant and NSAID nanosphere” and “NSAID nanosphere and Antioxidant nanosphere” as used herein refer to a nanosphere comprising molecules of Formula B-II and/or Formula B-III.

“Antioxidant nanosphere” as used herein refers to a nanosphere comprising molecules of Formula A-IV and/or A-V.

“NSAID/Antioxidant nanosphere combination” and “Antioxidant/NSAID nanosphere combination” as used herein refer to a nanosphere comprising a molecule selected from Formula B-I, B-II or B-III, and a molecule selected from Formula A-IV or A-V.

“NSAID nanosphere/Antioxidant nanosphere composition” and “Antioxidant nanosphere/NSAID nanosphere composition” as used herein refer to a composition comprising Antioxidant nanospheres in combination with NSAID nanospheres or Antioxidant and NSAID nanospheres.

As used herein, the term “aliphatic” means a moiety characterized by a straight or branched chain arrangement of constituent carbon atoms and can be saturated or partially unsaturated with one or more (e.g., one, two, three, four, five or more) double or triple bonds.

As used herein, the term “alicyclic” means a moiety comprising a nonaromatic ring structure. Alicyclic moieties can be saturated or partially unsaturated with one or more double or triple bonds. Alicyclic moieties can also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C₃-C₈ rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

As used herein, the term, “aromatic” means a moiety wherein the constituent atoms make up an unsaturated ring system, all atoms in the ring system are sp² hybridized and the total number of pi electrons is equal to 4n+2. An aromatic ring can be such that the ring atoms are only carbon atoms (e.g., aryl) or can include carbon and non-carbon atoms (e.g., heteroaryl).

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 1 and 6 carbons and at least one double bond, e.g., vinyl, allyl, 1-propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 1 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The terms “alkylene,” “alkenylene,” and “alkynylene” refer to divalent alkyl, alkelyne, and alkynylene” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula ═CRₐR_{b}. Cₓ alkylidene and Cₓ-C_{y}alkylidene are typically used where X and Y indicate the number of carbon atoms in the chain. For example, C₂-C₆alkylidene includes methylidene (═CH₂), ethylidene (═CHCH₃), isopropylidene (═C(CH₃)₂), propylidene (═CHCH₂CH₃), allylidene (═CH—CH═CH₂), and the like).

The term “heteroalkyl”, as used herein, refers to straight or branched chain, or cyclic carbon-containing radicals, or combinations thereof, containing at least one heteroatom. Suitable heteroatoms include, but are not limited to, 0, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl, 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y}aryl are typically used where X and Y indicate the number of carbon atoms in the ring system. Exemplary aryl groups include, but are not limited to, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. Heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring can be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cylcyl are typically used where X and Y indicate the number of carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like.

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻) ═, —O—, —S— or —S(O)₂—, —OS(O)₂—, and —SS—, wherein R^{N} is H or a further substituent.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—P—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term, “aromatic” means a moiety wherein the constituent atoms make up an unsaturated ring system, all atoms in the ring system are sp² hybridized and the total number of pi electrons is equal to 4n+2. An aromatic ring can be such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls (including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which can optionally also be substituted or unsubstituted. In some cases, two substituents, together with the carbon(s) to which they are attached to, can form a ring.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An“ether” is two hydrocarbons covalently linked by an oxygen. Accordingly, the substituent of an alkyl that renders that alkyl an ether is or resembles an alkoxyl, such as can be represented by one of —O-alkyl, —O— alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids, sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

It is noted in regard to all of the definitions provided herein that the definitions should be interpreted as being open ended in the sense that further substituents beyond those specified can be included. Hence, a C₁ alkyl indicates that there is one carbon atom but does not indicate what are the substituents on the carbon atom. Hence, a C₁ alkyl comprises methyl (i.e., —CH3) as well as CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

The term “derivative” as used herein refers to a chemical substance related structurally to another, i.e., an “original” substance, which can be referred to as a “parent” compound. A “derivative” can be made from the structurally-related parent compound in one or more steps. In some embodiments, the general physical and chemical properties of a derivative can be similar to or different from the parent compound.

### Nanosphere Comprising NSAIDs

In certain embodiments, the nanospheres are formed with hydrophobic NSAID derivatives. Thus, in certain embodiments, the nanospheres comprise hydrophobic NSAID derivatives. In certain embodiments, the nanospheres are formed with hydrophobic antioxidant and anti-inflammatory derivatives of an NSAID. Thus, in certain embodiments, the nanospheres comprise hydrophobic antioxidant and anti-inflammatory derivatives of an NSAID.

International Application Publication No. WO2009/148698 provides examples of hydrophobic NSAID derivatives and hydrophobic antioxidant and anti-inflammatory derivatives of an NSAID, and is incorporated herein by reference as though fully set forth in its entirety.

In various embodiments, the nanospheres are antioxidant nanospheres.

In certain embodiments, the nanospheres are formed with tocopherol. Thus, in certain embodiments, the nanospheres comprise tocopherol.

### NSAID Derivatives and Nanospheres

Various embodiments of the present invention use NSAID nanospheres comprising a hydrophobic derivative of an NSAID (“NSAID derivative”). In one embodiment, the nanosphere comprising NSAID of the present invention are capable of releasing the NSAID derivatives during a prolonged period of time, and thus reduce adverse gastrointestinal side effects caused by NSAIDs, while it is used for treating conditions, such as traumatic brain injury.

The NSAID nanospheres comprise derivatives of NSAIDs (“NSAID derivative”). In some embodiments, hydrophobic NSAID derivatives can be represented by Formula B-I:

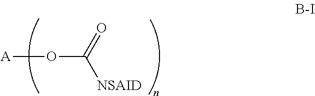

wherein the A is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; and n is an integer of at least two, and in particular embodiments n can be an integer from 2-4.

### Antioxidant and Anti-Inflammatory Derivatives and Nanospheres

Various embodiments of the present invention use antioxidant and NSAID nanospheres. In one embodiment, antioxidant and NSAID nanospheres are capable of releasing the NSAIDs during a prolonged period of time. Various embodiments of the present invention use hydrophobic antioxidant and anti-inflammatory derivatives of an NSAID. In some embodiments, the hydrophobic antioxidant and anti-inflammatory derivatives of an NSAID can be represented by Formula B-II:

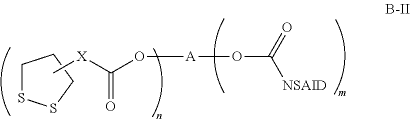

wherein X is selected from the group consisting of a substituted, unsubstituted, branched or unbranched chain of carbon atoms and can optionally contain a heteroatom; A is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; n is an integer of at least one; and m is an integer of at least one.

In some embodiments, the [1,2]-dithiolane moieties are from α-lipoic acid (“ALA”), and thus, the antioxidant and NSAID derivatives of the present invention can be represented by Formula B-III:

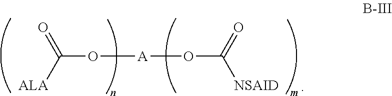

Accordingly, the antioxidant and NSAID nanospheres comprise a derivative of an NSAID and an α-lipoic acid.

Exemplary branched or unbranched alkyl for A in molecules of formula B-I, B-II or B-III include, but are not limited to, C1-2 alkyl, C1-3 alkyl, C1-4 alkyl, C1-6 alkyl, C1-8 alkyl, C1-10 alkyl or C1-12 alkyl. In some embodiments, A is a branched or unbranched alkyl comprising one, two, three, four, five, six, seven, eight, nine, ten, eleven, twelve or more carbons.

Exemplary branched or unbranched alkenyl for A in molecules of formula B-I, B-II or B-III include, but are not limited to, C2-3 alkenyl, C2-4 alkenyl, C2-6 alkenyl, C2-8 alkenyl, C2-10 alkenyl or C2-12 alkenyl. In some embodiments, A is a branched or unbranched alkenyl comprising two, three, four, five, six, seven, eight, nine, ten, eleven, twelve or more carbons.

Exemplary branched or unbranched alkynyl for A in molecules of formula B-I, B-II or B-III include, but are not limited to, C2-3 alkynyl, C2-4 alkynyl, C2-6 alkynyl, C2-8 alkynyl, C2-10 alkynyl or C2-12 alkynyl. In some embodiments, A is a branched or unbranched alkynyl comprising two, three, four, five, six, seven, eight, nine, ten, eleven, twelve or more carbons.

When the A is an heteroatom-containing branched or unbranched alkyl, heteroatom-containing branched or unbranched alkenyl, or heteroatom-containing branched or unbranched alkynyl, each heteroatom can be selected independently from N, O, and S. Accordingly, in some embodiments, the heteroatom is N. In some embodiments, heteroatom is O. In some embodiments, the heteroatom is S.

Exemplary cyclic aliphatic for A in molecules of formula B-I, B-II or B-III include, but are not limited to C3-12 cyclic aliphatic. For example, the cyclic aliphatic can be a C3, C4, C5, C6, C7, or C8 cyclic aliphatic. In some embodiments, the cyclic aliphatic is C8-12 cyclic aliphatic.

Exemplary cyclic aromatics for A in molecules of formula B-I, B-II or B-III include, but are not limited to C4-12 cyclic aromatics. For example, the cyclic aromatic can be a C4, C5, C6, C7, or C8 cyclic aromatic. In some embodiments, the cyclic aromatic is C8-12 cyclic aromatic.

Exemplary heterocyclic for A in molecules of formula B-I, B-II or B-III include, but are not limited to C4-12 heterocyclic. For example, the cyclic aromatic can be a C4, C5, C6, C7, or C8 heterocyclic. In some embodiments, the heterocyclic is C8-12 cyclic heterocyclic.

In various embodiments, A in molecules of formula B-I, B-II or B-III can be a polyol or a moiety that is formed by esterification of at least two free esterifiable hydroxyl groups on a polyol. In some embodiments, the polyol can be HO(CH₂CH₂O)ₙH, wherein n on the polyol can be an integer between 1 and 6. In some embodiments, the polyol can be HO(CH₂)ₙOH, wherein n on the polyol can be an integer between 3 and 16.

In other embodiments, A in molecules of formula B-I, B-II or B-III can be or formed from esterification of a polyol selected from group consisting of an ethylene glycol, diethylene glycol, triethylene glycol, tetraethylene glycol, pentaethylene glycol, hexaethylene glycol, 1,3-propanediol, and 1,4-butanediol. In some embodiments, the polyol can be selected from the commercial available polyols as shown in Table 1.

TABLE 1
Some exemplary polyols.

| Com- | |
|---|---|
| pound | Structure |
| 1 | 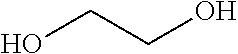 |
| 2 | 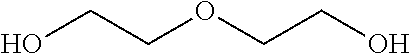 |
| 3 | 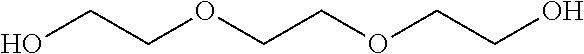 |
| 4 | 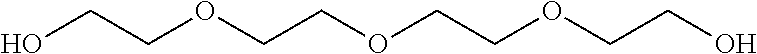 [SMILES: OCCOCCOCCOCCO] |
| 5 | 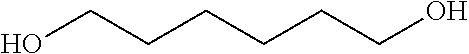 |
| 6 | 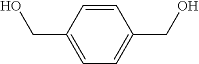 1,4-Benzenedimethanol |
| 7 | 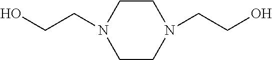 [SMILES: OCCN1CCN(CCO)CC1] 1,2-Bis(2-hydroxyethyl)-piperazine |
| 8 | 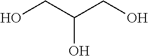 |
| 9 | 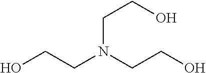 Triethanolamine |
| 10 | 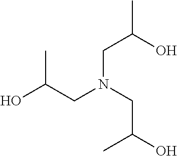 Triisopropanolamine |
| 11 | 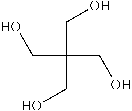 |
| 12 | 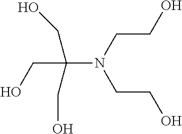 [SMILES: OCCN(CCO)C(CO)(CO)CO] |
| 13 | 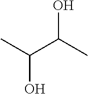 |
| 14 | 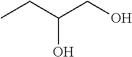 |
| 15 | 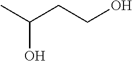 |
| 16 | 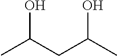 |
| 17 | 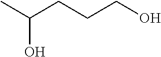 |
| 18 | 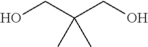 |
| 19 | 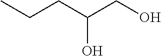 |
| 20 | 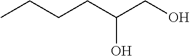 |
| 21 | 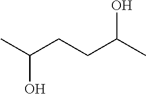 |
| 22 | 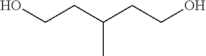 |
| 23 | 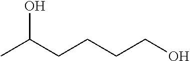 |
| 24 | 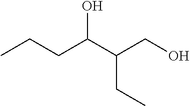 |
| 25 | 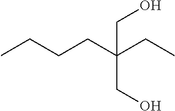 |
| 26 | 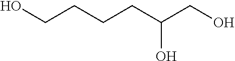 |
| 27 | 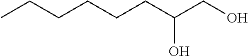 |
| 28 | 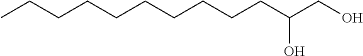 |
| 29 | 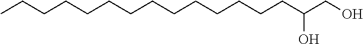 [SMILES: CCCCCCCCCCCCCCC(O)CO] |
| 30 | 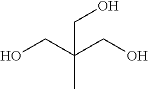 |
| 31 | 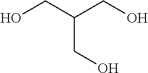 |
| 32 | 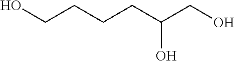 |
| 33 | 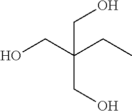 |
| 34 | 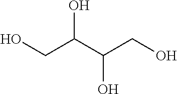 |
| 35 | 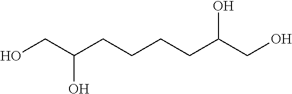 [SMILES: OCC(O)CCCCC(O)CO] |
| 36 | 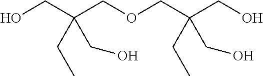 [SMILES: CCC(CO)(CO)COCC(CC)(CO)CO] |
| 37 | 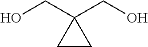 |
| 38 | 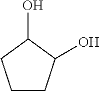 |
| 39 | 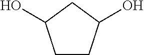 1,3,-Cyclopentanediol |
| 40 | 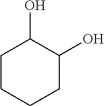 |
| 41 | 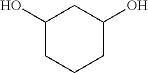 |
| 42 | 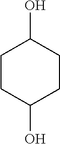 1,4-Cyclohexamediol |
| 43 | 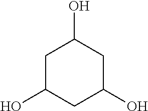 |
| 44 | 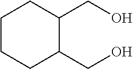 |
| 45 | 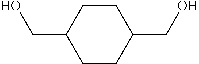 |
| 46 | 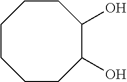 |
| 47 | 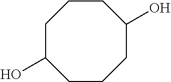 |
| 48 | 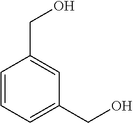 |
| 49 | 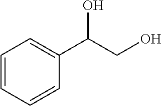 |
| 50 | 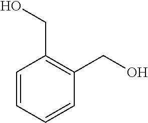 |
| 51 | 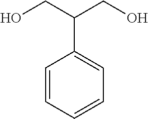 [SMILES: OCC(CO)c1ccccc1] |
| 52 | 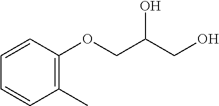 |
| 53 | 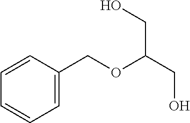 [SMILES: OCC(CO)OCc1ccccc1] |
| 54 | 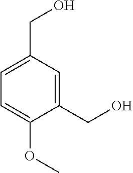 |
| 55 | 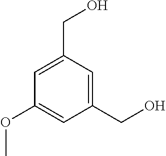 |
| 56 | 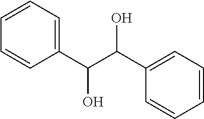 [SMILES: OC(c1ccccc1)C(O)c1ccccc1] |
| 57 | 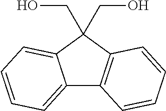 [SMILES: OCC1(CO)c2ccccc2-c2ccccc21] |
| 58 | 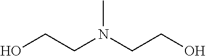 |
| 59 | 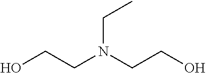 |
| 60 | 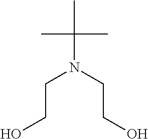 |
| 61 | 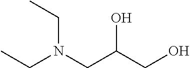 |
| 62 | 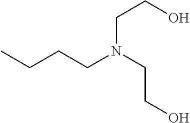 |
| 63 | 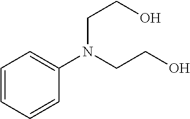 [SMILES: OCCN(CCO)c1ccccc1] |
| 64 | 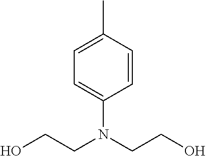 |
| 65 | 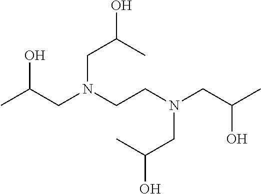 [SMILES: CC(O)CN(CCN(CC(C)O)CC(C)O)CC(C)O] |

In some embodiments, A in molecules of formula B-I, B-II or B-III can be a diamine or a moiety formed by using a diamine. In some embodiments, the diamines can NH₂—X—NH₂, wherein X can be a hydrocarbon group (for example, an alkyl, aryl, cycloaliphatic or aralkyl group), and can be saturated or unsaturated. X can also contain hetero atoms (e.g., nitrogen, oxygen, sulfur, etc.). In some other embodiments, the diamine can be NH₂(CH₂CH₂O)ₙCH₂CH₂NH₂, wherein n is an integer between 1 and 100. In still some other embodiments, diamine can be NH₂(CH₂)ₙNH₂, wherein n is an integer between 2 and 12.

In some embodiments, A in molecules of formula B-I, B-II or B-III can be an aminoalcohol or a moiety formed by using a aminoalcohol as the linker in the process of producing the NSAID derivative. Aminoalcohols that are useful in the present invention can include, but are not limited to, NH₂—Y—OH, wherein Y can be a hydrocarbon group; for example, an alkyl, aryl, cycloaliphatic or aralkyl group; and can be saturated or unsaturated. Y can also contain hetero atoms (e.g., nitrogen, oxygen, sulfur, etc.).

In some other embodiments, the aminoalcohol can be HO(CH₂CH₂O)ₙCH₂CH₂NH₂, wherein n is an integer between 1 and 100. In still some other embodiments, the aminoalcohol can be HO(CH₂)—NH₂, wherein n is an integer between 2 and 12.

### Non-Steroidal Anti-Inflammatory Drugs

Non-steroidal anti-inflammatory drugs (NSAIDs) are a promising candidate for controlling the deleterious effects of inflammation after TBI. Post injury inflammation leads to degradation of the blood brain barrier, edema, increased intracranial pressure, metabolic disturbances, activation of microglia and infiltration of peripheral immune cells [32-35]. These immune cells produce reactive oxygen species, which are especially damaging to the lipid rich membranes of the nervous system [36,37]. Injury induced inflammation also leads to several deleterious effects on cerebral blood vessels [38,39].

NSAIDs possess well-documented analgesic, antipyretic, and anti-inflammatory effects [40]. However, diffuse distribution of NSAIDs throughout the body leads to an array of adverse side effects, thought to be caused by free carboxylic acid groups and blockage of prostaglandin synthesis in the gastrointestinal system [41]. In order to circumvent the adverse side effects associated with NSAIDs and improve bioavailability, various NSAID prodrugs have been developed that mask carboxylic acid groups through the formation of bioreversible bonds [42-44].

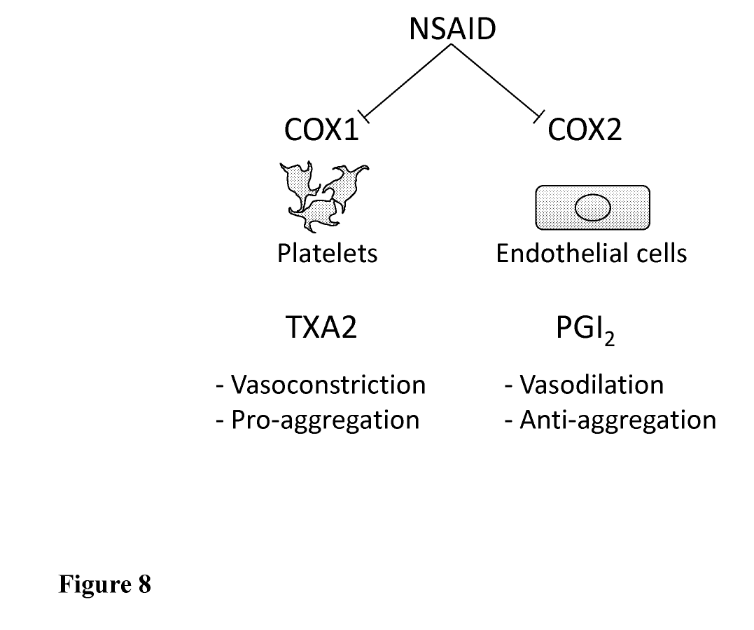

NSAIDs have widely known analgesic, antipyretic, and anti-inflammatory effects. Their mechanism of effect is through COX inhibition. COX enzymes are produced as two isoforms, COX-1 and COX-2. In endothelial tissue, the constitutive production of COX-2 leads to the production of PGI₂, which causes vasorelaxation and inhibits platelet aggregation. Normal hemostasis is maintained by a balance between this epithelial effect and a COX-1 catalyzed thromboxane A2 activity in platelets, which mediates a vasoconstrictive and pro-aggregation effect [66] (FIG. 8). Therefore, our use of a non-selective COX inhibitor would be expected to better maintain a natural hemostatic balance.

In the brain, COX-2 induction is known to be upregulated after traumatic brain injury in rats starting at 3 hours and lasting for at least 12 days [67]. Such elevated production of COX-2 is thought to increase cellular damage, vascular dysfunction, and alterations in cellular metabolism [68]. COX-2 catalyzed production of prostaglandin PGE₂ results in the production of free radicals. Free radical-induced lipid peroxidation is responsible for massive neuronal death following primary mechanical injury [69], and PGE₂ itself is also neurotoxic [70,71]. In the short term, vascular permeability in response to inflammatory cell signaling leads to edema and intracranial hypertension, which further contributes to cell death [63,72]. In the long term, inflammation has been linked with the development of numerous neurodegenerative diseases including amyotrophic lateral sclerosis, multiple sclerosis, Alzheimer's Disease, and Parkinson's Disease [9,49,73,74].

The NSAID can be a non-steroidal anti-inflammatory drug containing a carboxylic acid. NSAIDs are well known in the art and one of skill in the art will be able to readily choose an NSAID without undue experimentation. The carboxylic group of the NSAIDs is temporarily masked via hydrolysable bond, and can therefore act as a prodrug and reduce the side effect and also has advantage in the controlled and sustained release of the drugs.

Examples of NSAIDs include, but are not limited to, aspirin, ibuprofen, flurbiprofen, ketoprofen, fenoprofen, fenbufen, naproxen, indomethacin, diclofenac, ketorolac, tolmetin, flufenamic acid, mefenamic acid, tolfenamic acid, meclofenamic acid, niflumic acid, sulindac, and sulindac sulfide. Structures of some exemplary NSAID are shown below.

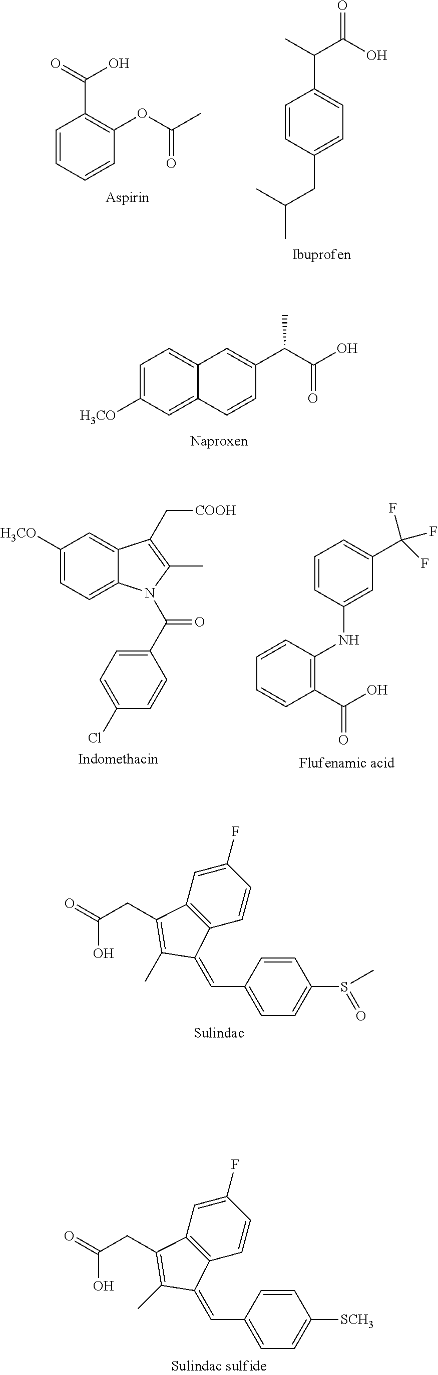

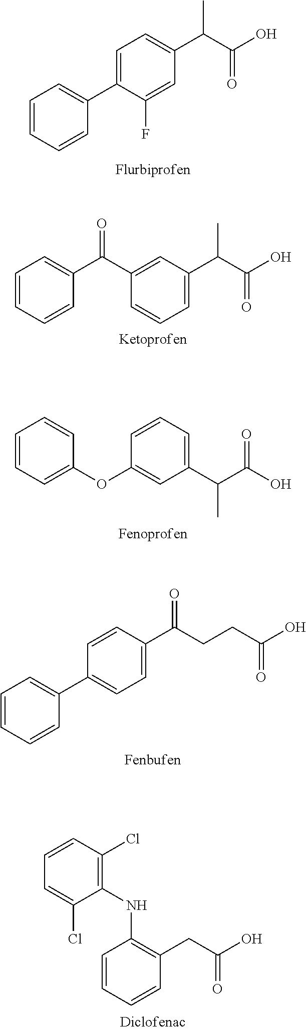 [SMILES: CC(C(=O)O)c1ccc(-c2ccccc2)c(F)c1 ; CC(C(=O)O)c1cccc(C(=O)c2ccccc2)c1 ; CC(C(=O)O)c1cccc(Oc2ccccc2)c1 ; O=C(O)CCC(=O)c1ccc(-c2ccccc2)cc1 ; O=C(O)Cc1ccccc1Nc1c(Cl)cccc1Cl]

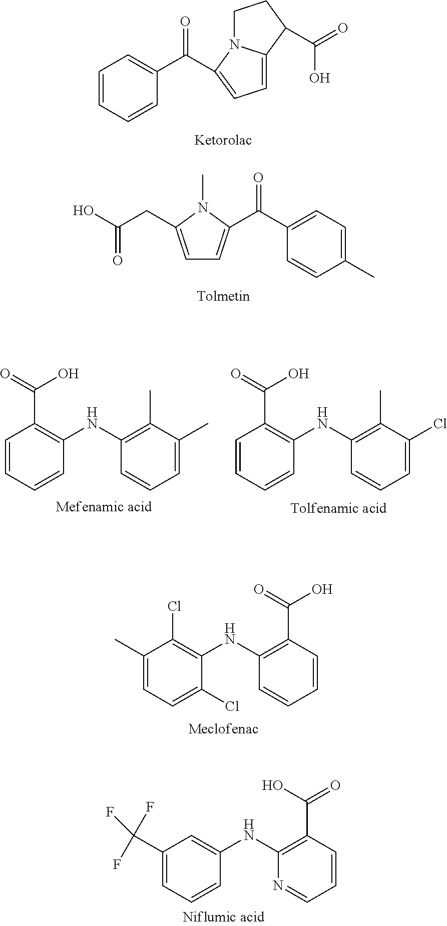

In some embodiments, the NSAIDs in compounds of formula B-I, B-II, or B—III can be selected from the group consisting of aspirin, ibuprofen, flurbiprofen, ketoprofen, fenoprofen, fenbufen, naproxen, indomethacin, diclofenac, ketorolac, tolmetin, flufenamic acid, mefenamic acid, tolfenamic acid, meclofenamic acid, niflumic acid, sulindac, sulindac sulfide, and any combinations thereof.

### Exemplary Hydrophobic Derivatives of NSAIDs

Examples of useful hydrophobic derivatives of NSAIDs, for example compounds of formula B-I, include, but are not limited to, those represented by formulas as follows:

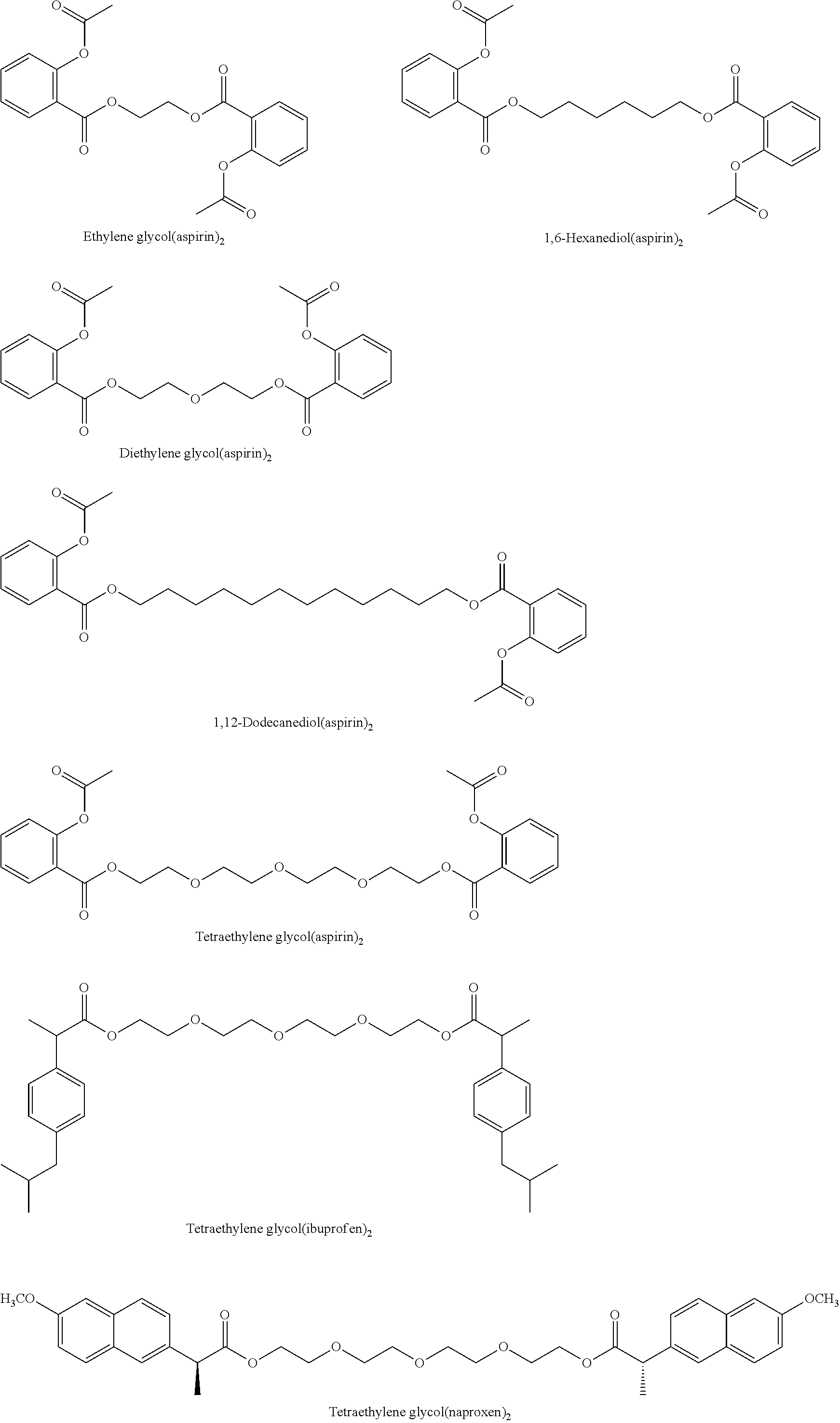

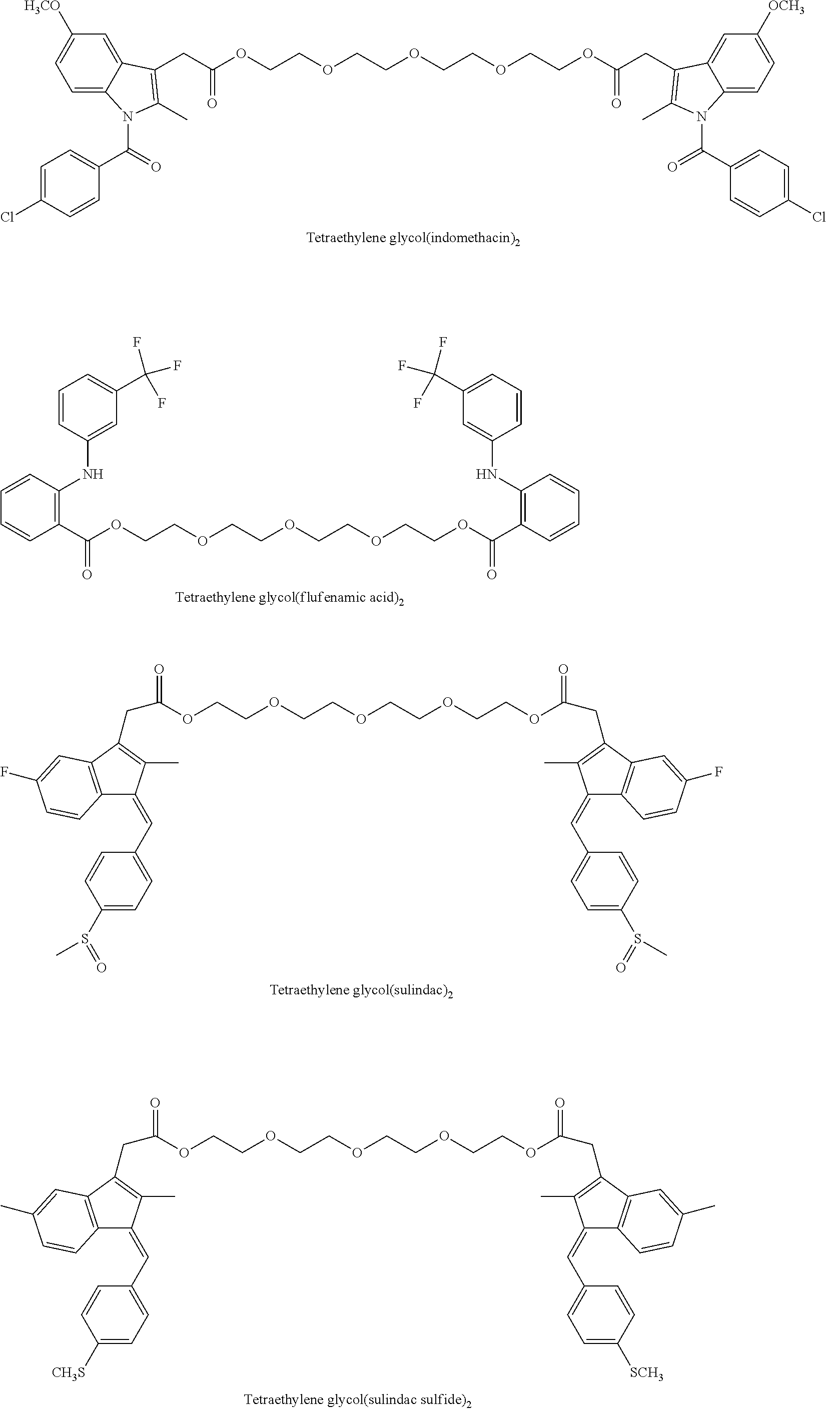

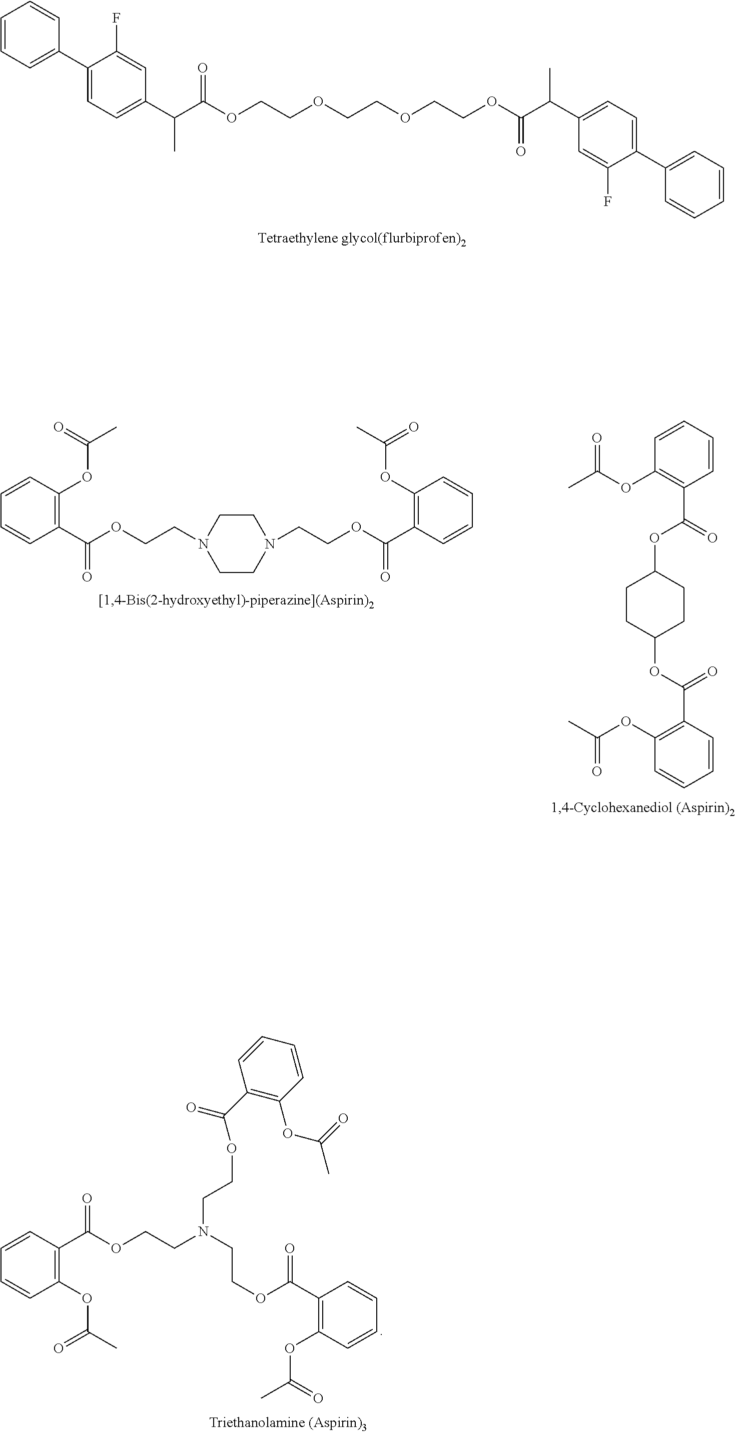 [SMILES: CC(C(=O)OCCOCCOCCOC(=O)C(C)c1ccc(-c2ccccc2)c(F)c1)c1ccc(-c2ccccc2)c(F)c1 ; CC(=O)Oc1ccccc1C(=O)OCCN1CCN(CCOC(=O)c2ccccc2OC(C)=O)CC1 ; CC(=O)Oc1ccccc1C(=O)OC1CCC(OC(=O)c2ccccc2OC(C)=O)CC1 ; CC(=O)Oc1ccccc1C(=O)OCCN(CCOC(=O)c1ccccc1OC(C)=O)CCOC(=O)c1ccccc1OC(C)=O]

A general scheme for the synthesis of the multiple NSAID-containing hydrophobic compounds and preparation of the nanospheres comprising NSAID are described in WO2009/148698, herein incorporated by reference as though fully set forth in its entirely, and the ensuing examples. The nanospheres showed sustained release of the free NSAIDs upon enzymatic hydrolysis by esterase.

### Exemplary Hydrophobic Antioxidant and NSAID Derivatives

Examples of useful hydrophobic antioxidant and NSAID derivatives, e.g., compounds of formula B-II or B-III, include, but are not limited to, those represented by formulas as follows:

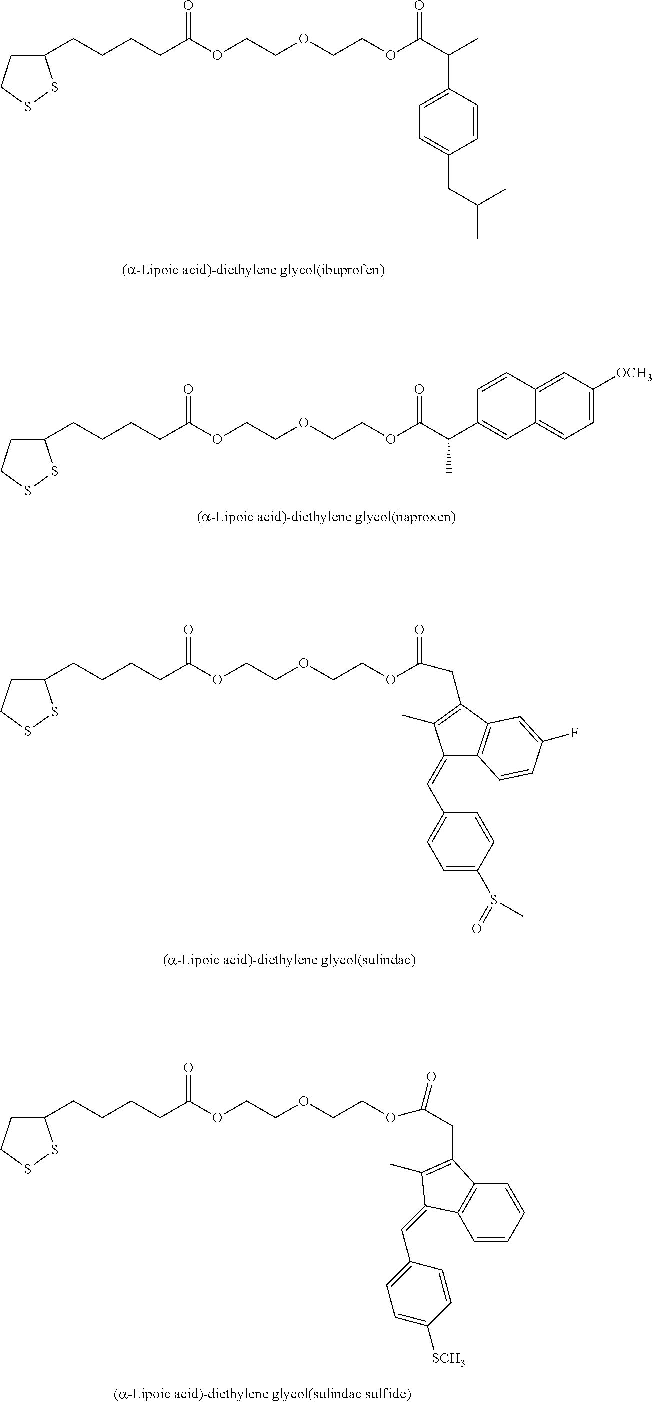 [SMILES: CC(C)Cc1ccc(C(C)C(=O)OCCOCCOC(=O)CCCCC2CCSS2)cc1 ; COc1ccc2cc([C@H](C)C(=O)OCCOCCOC(=O)CCCCC3CCSS3)ccc2c1 ; CC1=C(CC(=O)OCCOCCOC(=O)CCCCC2CCSS2)c2cc(F)ccc2/C1=C\c1ccc(S(C)=O)cc1 ; CSc1ccc(/C=C2/C(C)=C(CC(=O)OCCOCCOC(=O)CCCCC3CCSS3)c3ccccc32)cc1]

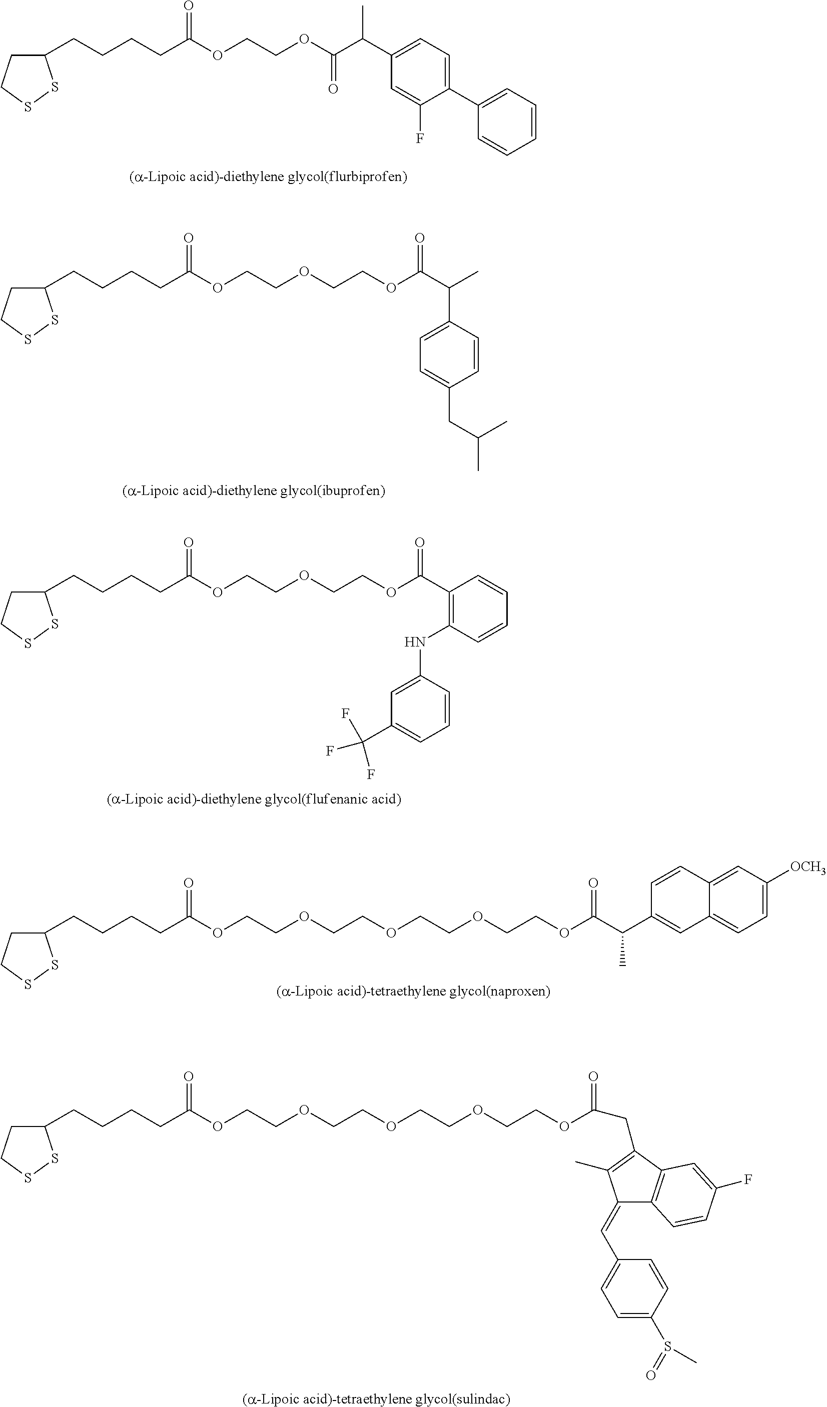 [SMILES: CC(C(=O)OCCOC(=O)CCCCC1CCSS1)c1ccc(-c2ccccc2)c(F)c1 ; CC(C)Cc1ccc(C(C)C(=O)OCCOCCOC(=O)CCCCC2CCSS2)cc1 ; O=C(CCCCC1CCSS1)OCCOCCOC(=O)c1ccccc1Nc1cccc(C(F)(F)F)c1 ; COc1ccc2cc([C@H](C)C(=O)OCCOCCOCCOCCOC(=O)CCCCC3CCSS3)ccc2c1 ; CC1=C(CC(=O)OCCOCCOCCOCCOC(=O)CCCCC2CCSS2)c2cc(F)ccc2/C1=C\c1ccc(S(C)=O)cc1]

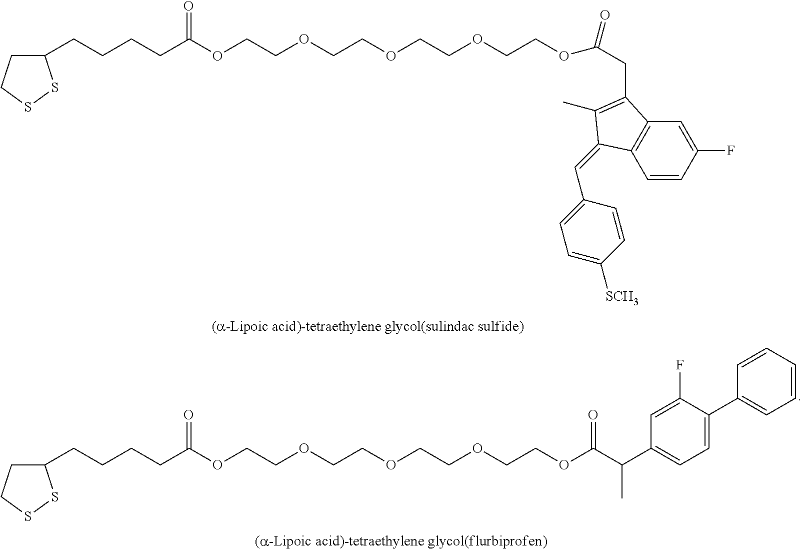 [SMILES: CSc1ccc(/C=C2/C(C)=C(CC(=O)OCCOCCOCCOCCOC(=O)CCCCC3CCSS3)c3cc(F)ccc32)cc1 ; CC(C(=O)OCCOCCOCCOCCOC(=O)CCCCC1CCSS1)c1ccc(-c2ccccc2)c(F)c1]

### Antioxidants

In some embodiments, the nanosphere further comprises an antioxidant. In some embodiments, the antioxidant is tocopherol, e.g., α-Tocopherol. α-Tocopherol is the most biologically active form of vitamin E and is believed to be the most potent lipid-soluble antioxidant because it is capable of breaking the chain of propagation of free radical mediated lipid peroxidation [45,46]. Oxidative damage caused by reactive oxygen species (ROS) is believed to be a major feature in the pathophysiology of many neurodegenerative diseases [37,47]. Various studies suggest that long-term use of NSAIDs can prevent or delay dementia in Alzheimer's Disease, which is characterized by an increased inflammatory profile in the brain similar to TBI [48-51].

In some embodiments, the nanoprodrug of the invention contains α-tocopherol (vitamin E) as an antioxidant component and stabilizing structural component. Increasing evidence suggests that vitamin E can play a promising role in the prevention and treatment of oxidative damage-related neurodegenerative diseases [37,47,75,76]. However, its extreme insolubility in water poses a serious limitation to distribution in the aqueous biological environment, limiting its usefulness as a therapeutic intervention. Efforts to make α-tocopherol more water soluble by replacing the lipophilic phytyl chain with more hydrophilic moieties interfere with its antioxidant capabilities and can incur unexpected adverse biological effects [46,77]. Thus, unmodified α-tocopherol was used in the formulation of the nanoprodrug as a stabilizing and size reducing structural component, in addition to its antioxidant benefits. Despite the hydrophobicity of Ibu₂TEG and α-tocopherol alone, formation of the two into a nanoparticle generates a large surface area for hydrolytic esterase enzymes to interact and degrade prodrugs, releasing ibuprofen from the surface [61].

The blood brain barrier (BBB) is a tightly regulated interface between the central nervous system and the circulating blood, formed by CD31+ vascular endothelial tissue. The BBB protects the CNS from edema and neurotoxic macromolecules. When the BBB is functioning normally, it also often blocks the delivery of therapeutics that would be used to treat conditions such as neurodegenerative diseases, CNS infections, and brain tumors. However, in TBI the integrity of the BBB is known to be severely compromised at the site of injury [52,53]. The destruction of the BBB interface can be a direct result of the traumatic injury itself, as well as due to secondary consequences of inflammation-related mechanisms, metabolic disturbances, and astrocyte dysfunction. This permeability can represent a serendipitous opportunity to deliver drugs to the site of injury [54].

The phenomenon of failing vascular barrier activity has been described in oncology literature as the enhanced permeability and retention (EPR) effect [55,56]. Many rapidly growing solid tumor types exhibit defects in angiogenesis, resulting in the formation of poorly organized and highly permeable blood vessel structures. Although the etiology for the EPR effect is different in TBI than it is in tumor formation, the effect is the same [52,57,58]. Thus, it seems appropriate to describe vascular permeability in TBI using the same term. Just as the EPR effect has been shown to permit an increase in chemotherapeutic nanodrug uptake in tumors, it has also been recently shown to allow proteins chaperoned by polybutyl cyanoacrylate to be delivered to injured tissue in TBI using rats [59].

The inflammatory response cascade after traumatic brain injury contributes to secondary damage and degeneration as well as post injury repair [38,53,62,63]. Because the cyclooxygenase (COX) system is ubiquitously expressed in the body, previous attempts to treat TBI with COX inhibitors have been confounded by off target effects. Such side effects are particularly dangerous to trauma patients with TBI because of their increased risk for stress ulcer formation and subsequent GI hemorrhage [64]. In this study, the inventors used a recently developed hydrophobic derivative of the non-selective COX-1 and 2 inhibitor ibuprofen, combined with the antioxidant α-tocopherol and 1-octadecanethiol which binds the Cy5.5 fluorescent tracer. While this exemplary nanosphere comprises 1-octadecanethiol binding to the Cy5.5 fluorescent tracer, therapeutic embodiments without 1-octadecanethiol or 1-octadecanethiol binding to the Cy5.5 fluorescent tracer will also have the same therapeutic effect. Each pair of ibuprofen molecules is joined by tetra ethylene glycol (TEG), forming Ibu₂TEG. In Ibu₂TEG, the carboxylic acid functional groups of the ibuprofens are esterified upon joining to TEG. Not only does this protect the ibuprofen from premature degradation, but it can protect off target tissues from irritation by the acidic carboxylic acid groups. At the region of injury, where the blood brain barrier has increased permeability, we found significant accumulation of the nanoprodrug. Our finding has significant clinical implications as a potential treatment for TBI that is both safe and effective.

Various embodiments of the present invention provide for methods of using the nanospheres described herein. Various embodiments of the present invention provide for methods of using the nanospheres comprising a therapeutic agent or diagnostic agent on an amphiphilic spacer describe herein. Various embodiments of the present invention provide for methods of using nanospheres comprising a therapeutic agent or a diagnostic agent on an amphiphilic polymer as described herein. Methods of using these nanospheres include administering a nanosphere of the present invention to a subject in need of treatment for traumatic brain injury.

### Therapeutic Agents

In some embodiments, the nanosphere further comprises a therapeutic agent. Accordingly, the therapeutic agent is delivered to the TBI tissue when the nanosphere is delivered to the TBI tissue. The delivery of the therapeutic agent to the TBI tissue treats TBI.

In some embodiments, the therapeutic agent can be conjugated to a hydrophilic spacer, a hydrophobic spacer, an amphiphilic spacer, or an amphiphilic polymer. In various embodiments, the therapeutic agent is a NSAID. The NSAID can be selected from the group consisting of aspirin, ibuprofen, flurbiprofen, ketoprofen, fenoprofen, fenbufen, naproxen, indomethacin, diclofenac, ketorolac, tolmetin, flufenamic acid, mefenamic acid, tolfenamic acid, meclofenamic acid, niflumic acid, sulindac, sulindac sulfide and combinations thereof.

In some embodiments, the therapeutic agent is a statin. Exemplary statins include, but are not limited to atorvastatin, cerivastatin, fluvastatin, lovastatin, lactones of lovastatin, mevastatin, pitavastatin, pravastatin, rosuvastatin, velostatin, rivastatin, itavastatin, simvastatin, and lactones thereof.

In some embodiments, the therapeutic agent is an antioxidant.

In some embodiments, the therapeutic agent is erythropoietin, peptide, antisense nucleic acid, DNA, RNA, or protein.

It is appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It is appreciated that the therapies employed can achieve a desired effect for the same disorder (for example, an inventive compound can be administered concurrently with another TBI therapeutic agent), or they can achieve different effects (e.g., control of an adverse effects). In some embodiments, the nanospheres comprising NSAIDs can be given in combination with diuretics, anti-seizure drugs, and/or coma-inducing drugs.

Nanospheres Prepared from Mixture of the Inventive NSAID Derivatives and Polymers and/or Oils

Various embodiments of the present invention also provide for a nanosphere comprising an NSAID derivative and a polymer and/or oily product. The NSAID derivatives can be ones as described above. Examples of polymers include, but not limited to, polyanhydrides, polyesters, polyorthoesters, polyesteramides, polyacetals, polyketals, polycarbonates, polyphosphoesters, polyphosphazene, polyvinylpyrrolidone, polydioxanones, poly(malic acid), poly(amino acids), polymers of N-2-(hydroxypropyl)methacrylamide (HPMA), polymers of N-isopropyl acrylamide (NIPAAm), polyglycolide, polylactide, copolymers of glycolide and lactide (e.g., poly(lactide-co-glycolide), and blends thereof. Examples of oily products include, but not limited to, vegetable oils, mineral oils, vitamins, esters of carboxylic acids and combinations thereof.

NSAID Nanospheres Combined with Antioxidant Nanospheres

Various embodiments of the present invention also provide for a composition comprising Antioxidant nanospheres in combination with NSAID nanospheres or Antioxidant and NSAID nanospheres (“NSAID nanosphere/antioxidant nanosphere composition”). The NSAID nanospheres and the Antioxidant and NSAID nanospheres can be ones as described above. The antioxidant nanospheres can be ones as described in International Application No. PCT/US08/88541, incorporated herein by references as though fully set forth.

Briefly, the antioxidant nanospheres comprise an antioxidant molecule represented by the Formula A-IV:

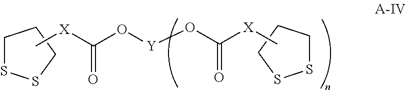

wherein X is selected from the group consisting of a substituted, unsubstituted, branched or unbranched chain of carbon atoms and can optionally contain a heteroatom; Y is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; and n is an integer and is at least one.

In some embodiments, the [1,2]-dithiolane moieties in molecules of formula A-IV can be independently from α-lipoic acid, and the antioxidants molecules are generally represented by the formula A-V:

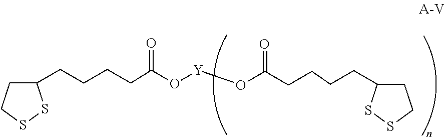

In this embodiment, at least two α-lipoic acids are linked to a polyol via ester bonds.

Exemplary branched or unbranched alkyl for X in molecules of formula A-IV or A-V include, but are not limited to, C1-2 alkyl, C1-3 alkyl, C1-4 alkyl, C1-6 alkyl, C1-8 alkyl, C1-10 alkyl or C1-12 alkyl. In some embodiments, A is a branched or unbranched alkyl comprising one, two, three, four, five, six, seven, eight, nine, ten, eleven, twelve or more carbons.

Exemplary branched or unbranched alkenyl for X in molecules of formula BA-IV or A-V include, but are not limited to, C2-3 alkenyl, C2-4 alkenyl, C2-6 alkenyl, C2-8 alkenyl, C2-10 alkenyl or C2-12 alkenyl. In some embodiments, A is a branched or unbranched alkenyl comprising two, three, four, five, six, seven, eight, nine, ten, eleven, twelve or more carbons.

Exemplary branched or unbranched alkynyl for X in molecules of formula A-IV or A-V include, but are not limited to, C2-3 alkynyl, C2-4 alkynyl, C2-6 alkynyl, C2-8 alkynyl, C2-10 alkynyl or C2-12 alkynyl. In some embodiments, A is a branched or unbranched alkynyl comprising two, three, four, five, six, seven, eight, nine, ten, eleven, twelve or more carbons.

When X is an heteroatom-containing branched or unbranched alkyl, heteroatom-containing branched or unbranched alkenyl, or heteroatom-containing branched or unbranched alkynyl, each heteroatom can be selected independently from N, O, and S. Accordingly, in some embodiments, the heteroatom is N. In some embodiments, heteroatom is O. In some embodiments, the heteroatom is S.

Exemplary branched or unbranched alkyl for Y in molecules of formula A-IV or A-V include, but are not limited to, C1-2 alkyl, C1-3 alkyl, C1-4 alkyl, C1-6 alkyl, C1-8 alkyl, C1-10 alkyl or C1-12 alkyl. In some embodiments, A is a branched or unbranched alkyl comprising one, two, three, four, five, six, seven, eight, nine, ten, eleven, twelve or more carbons.

Exemplary branched or unbranched alkenyl for Y in molecules of formula BA-IV or A-V include, but are not limited to, C2-3 alkenyl, C2-4 alkenyl, C2-6 alkenyl, C2-8 alkenyl, C2-10 alkenyl or C2-12 alkenyl. In some embodiments, A is a branched or unbranched alkenyl comprising two, three, four, five, six, seven, eight, nine, ten, eleven, twelve or more carbons.

Exemplary branched or unbranched alkynyl for Y in molecules of formula A-IV or A-V include, but are not limited to, C2-3 alkynyl, C2-4 alkynyl, C2-6 alkynyl, C2-8 alkynyl, C2-10 alkynyl or C2-12 alkynyl. In some embodiments, A is a branched or unbranched alkynyl comprising two, three, four, five, six, seven, eight, nine, ten, eleven, twelve or more carbons.

When the Y is an heteroatom-containing branched or unbranched alkyl, heteroatom-containing branched or unbranched alkenyl, or heteroatom-containing branched or unbranched alkynyl, each heteroatom can be selected independently from N, O, and S. Accordingly, in some embodiments, the heteroatom is N. In some embodiments, heteroatom is O. In some embodiments, the heteroatom is S.

Exemplary cyclic aliphatic for Y in molecules of formula A-IV or A-V include, but are not limited to C3-12 cyclic aliphatic. For example, the cyclic aliphatic can be a C3, C4, C5, C6, C7, or C8 cyclic aliphatic. In some embodiments, the cyclic aliphatic is C8-12 cyclic aliphatic.

Exemplary cyclic aromatics for Y in molecules of formula A-IV or A-V include, but are not limited to C4-12 cyclic aromatics. For example, the cyclic aromatic can be a C4, C5, C6, C7, or C8 cyclic aromatic. In some embodiments, the cyclic aromatic is C8-12 cyclic aromatic.

Exemplary heterocyclic for Y in molecules of formula A-IV or A-V include, but are not limited to C4-12 heterocyclic. For example, the cyclic aromatic can be a C4, C5, C6, C7, or C8 heterocyclic. In some embodiments, the heterocyclic is C8-12 cyclic heterocyclic.

In various embodiments, Y in molecules of formula A-IV or A-V can be a polyol or a moiety that is formed by esterification of at least two free esterifiable hydroxyl groups on a polyol. In some embodiments, the polyol can be HO(CH₂CH₂O)ₙH, wherein n on the polyol can be an integer between 1 and 6. In some embodiments, the polyol can be HO(CH₂)ₙOH, wherein n on the polyol can be an integer between 3 and 16.

In other embodiments, Y in molecules of formula A-IV or A-V can be or formed from esterification of a polyol selected from group consisting of an ethylene glycol, diethylene glycol, triethylene glycol, tetraethylene glycol, pentaethylene glycol, hexaethylene glycol, 1,3-propanediol, and 1,4-butanediol. In some embodiments, Y in molecules of formula A-IV or A-V can be a polyol selected from those listed in Table 1.

In some embodiments, Y in molecules of formula A-IV or A-V can be a diamine or a moiety formed by using a diamine. In some embodiments, the diamines can NH₂—X—NH₂, wherein X can be a hydrocarbon group (for example, an alkyl, aryl, cycloaliphatic or aralkyl group), and can be saturated or unsaturated. X can also contain hetero atoms (e.g., nitrogen, oxygen, sulfur, etc.). In some other embodiments, the diamine can be NH₂(CH₂CH₂O)ₙCH₂CH₂NH₂, wherein n is an integer between 1 and 100. In still some other embodiments, diamine can be NH₂(CH₂)ₙNH₂, wherein n is an integer between 2 and 12.

In some embodiments, Y in molecules of formula A-IV or A-V can be an aminoalcohol or a moiety formed by using a aminoalcohol as the linker in the process of producing the NSAID derivative. Aminoalcohols that are useful in the present invention can include, but are not limited to, NH₂—Z—OH, wherein Z can be a hydrocarbon group; for example, an alkyl, aryl, cycloaliphatic or aralkyl group; and can be saturated or unsaturated. Y can also contain hetero atoms (e.g., nitrogen, oxygen, sulfur, etc.). In some other embodiments, the aminoalcohol can be HO(CH₂CH₂O)ₙCH₂CH₂NH₂, wherein n is an integer between 1 and 100. In still some other embodiments, the aminoalcohol can be HO(CH₂)ₙNH₂, wherein n is an integer between 2 and 12.

### NSAID/Antioxidant Nanosphere Combination

Various embodiments of the present invention also provide for a nanosphere comprising a molecule selected from Formula B-I, B-II or B-III as described above, and a molecule selected from Formula A-IV or A-V as described above (“NSAID/antioxidant nanosphere combination”).

The molecules of Formula B-I, B-II or B-III and molecules of formula A-IV or A-V can be present in any desired ratio in the nanosphere. For example, the molecules can be in a ratio ranging from about 100:1 to 1:100. In some embodiments, the molecules can be in a ratio ranging from about 50:1 to 1:50, 25:1 to 1:25, 10:1 to 1:10, 5:1 to 1:5, or 2.5:1 to 1:2.5. In some embodiments, the molecules can be in a ratio of about 1:1.

### Amphiphilic Spacer

A hydrophilic or hydrophobic spacer used in the present disclosure is a molecule that comprises hydrophilic or hydrophobic parts in one molecule, and can further comprise chemically active functional group on one end or both ends which can be used as a carrier for a therapeutic agent, diagnostic agent, or another spacer by conjugating it with the therapeutic agent, diagnostic agent, or another spacer molecule.

In some embodiments, the nanosphere further comprises an amphiphilic spacer.

An amphiphilic spacer used in the present disclosure is a molecule that comprises both hydrophilic and hydrophobic parts in one molecule, and the hydrophilic part can further comprise chemically active functional group which can be used as a carrier for a therapeutic or diagnostic agent by conjugating it with the therapeutic agent or diagnostic agent. In various embodiments, the chemically active functional group can be selected from the group consisting of thiol, amine, carboxylic acid, carboxylic acid NHS ester, maleimide, hydrazine, ketone, and aldehyde. An amphiphilic spacer used in the present disclosure also can be made by conjugating a hydrophilic spacer with a hydrophobic spacer. The end of the hydrophilic part further comprises chemically active functional group which can be used as a carrier for a therapeutic or diagnostic agent by conjugating it with the therapeutic agent or diagnostic agent.

In various embodiments, the amphiphilic spacer comprises a hydrophobic part and hydrophilic part. In various embodiments, the hydrophobic part of amphiphilic spacer is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups, and combinations thereof.

In various embodiments, the hydrophilic part of amphiphilic spacer comprises a molecule selected from the group consisting of heteroatom-containing branched and unbranched alkenyl, heteroatom-containing branched and unbranched alkynyl, aryl, cyclic aliphatic, cyclic aromatic, heterocyclic, and aromatic heterocyclic groups, and a chemically active group selected from the group consisting of thiol, amine, carboxylic acid, carboxylic acid NHS ester, maleimide, hydrazine, ketone, aledehyde, and combinations thereof.

In various embodiments, the amphiphilic spacer comprises an alkylthiol. In various embodiments, the amphiphilic spacer is an alkylthiol. In some embodiments, the alkylthiol is C2-4alkylthiol. In some embodiments, the alkylthiol is C2-4alkylthiol. In some embodiments, the alkylthiol is C4-6alkylthiol. In some embodiments, the alkylthiol is C6-8alkylthiol. In some embodiments, the alkylthiol is C8-10alkylthiol. In some embodiments, the alkylthiol is C10-12alkylthiol. In some embodiments, the alkylthiol is C12-14alkylthiol. In some embodiments, the alkylthiol is C14-18alkylthiol. In some embodiments, the alkylthiol is C18-20alkylthiol. In some embodiments, the alkylthiol is C10-18alkylthiol. In some embodiments, the alkylthiol is C22-24alkylthiol. In some embodiments, the alkylthiol is C24-30alkylthiol. In various embodiments, the alkylthiol is a straight chain alkylthiol.

In various embodiments, the amphiphilic spacer is selected from a C10, C11, C12, C13, C14, C15, C16, C17, C18, C19, C20, C21, C22, C23, C24, C25, C26, C27, C28, C29, C30, C31, C32, C34, C35, C36, C37, C38, C39 and C40 straight chain alkylthiol. In some embodiments, the amphiphilic spacer is 1-octadecanethiol.

In various embodiments, the amphiphilic spacer comprises an alkylamine. In various embodiments, the amphiphilic spacer is an alkylamine. In some embodiments, the alkylamine is C2-4alkylamine. In some embodiments, the alkylamine is C2-4alkylamine. In some embodiments, the alkylamine is C4-6alkylamine. In some embodiments, the alkylamine is C6-8alkylamine. In some embodiments, the alkylamine is C8-10alkylamine. In some embodiments, the alkylamine is C10-12alkylamine. In some embodiments, the alkylamine is C12-14alkylamine. In some embodiments, the alkylamine is C14-18alkylamine. In some embodiments, the alkylamine is C18-20alkylamine. In some embodiments, the alkylamine is C10-18alkylamine. In some embodiments, the alkylamine is C22-24alkylamine. In some embodiments, the alkylamine is C24-30alkylamine. In some embodiments, the alkylamine is a straight chain alkylamine.

In various embodiments, the amphiphilic spacer is selected from a C10, C11, C12, C13, C14, C15, C16, C17, C18, C19, C20, C21, C22, C23, C24, C25, C26, C27, C28, C29, C30, C31, C32, C34, C35, C36, C37, C38, C39 and C40 straight chain alkylamine.

### Amphiphilic Polymer

In various embodiments, the amphiphilic polymer comprises a polymer backbone, a hydrophilic part of the polymer and a hydrophobic part of the polymer. In various embodiments, the polymer backbone can be from natural polymer, modified natural polymer, synthetic polymer, and combinations thereof.

In various embodiments, the polymer backbone is selected from the group consisting of a polyanhydride, polyester, polyorthoester, polyesteramide, polyacetal, polyketal, polycarbonate, polyphosphoester, polyphosphazene, polyvinylpyrrolidone, polydioxanone, poly(malic acid), poly(amino acid), polymer of N-2-(hydroxypropyl)methacrylamide (HPMA), polymer of N-isopropyl acrylamide (NIPAAm), polyglycolide, polylactide, copolymer of glycolide and lactide, and combinations thereof.

In various embodiments, the hydrophobic part of amphiphilic polymer is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups, and combinations thereof.

In various embodiments, the hydrophilic part of amphiphilic polymer comprises a molecule selected from the group consisting of heteroatom-containing branched and unbranched alkenyl, heteroatom-containing branched and unbranched alkynyl, aryl, cyclic aliphatic, cyclic aromatic, heterocyclic, and aromatic heterocyclic groups, and a chemically active group selected from the group consisting of thiol, amine, carboxylic acid, carboxylic acid NHS ester, maleimide, hydrazine, ketone, aledehyde, and combinations thereof.

In various embodiments, the amphiphilic polymer comprises an alkylthiol. In various embodiments, the amphiphilic spacer is an alkylthiol. In some embodiments, the alkylthiol is C2-4alkylthiol. In some embodiments, the alkylthiol is C2-4alkylthiol. In some embodiments, the alkylthiol is C4-6alkylthiol. In some embodiments, the alkylthiol is C6-8alkylthiol. In some embodiments, the alkylthiol is C8-10alkylthiol. In some embodiments, the alkylthiol is C10-12alkylthiol. In some embodiments, the alkylthiol is C12-14alkylthiol. In some embodiments, the alkylthiol is C14-18alkylthiol. In some embodiments, the alkylthiol is C18-20alkylthiol. In some embodiments, the alkylthiol is C10-18alkylthiol. In some embodiments, the alkylthiol is C22-24alkylthiol. In some embodiments, the alkylthiol is C24-30alkylthiol. In some embodiments, the alkylthiol is a straight chain alkylthiol. In various embodiments, the amphiphilic polymer is selected from a C2, C3, C4, C6, C7, C8, C9, C10, C11, C12, C13, C14, C15, C16, C17, C18, C19, C20, C21, C22, C23, C24, C25, C26, C27, C28, C29, C30, C31, C32, C34, C35, C36, C37, C38, C39 and C40 straight chain alkylthiol.

In various embodiments, the amphiphilic polymer comprises an alkylamine. In various embodiments, the amphiphilic polymer is an alkylamine. In some embodiments, the alkylamine is C2-4alkylamine. In some embodiments, the alkylamine is C2-4alkylamine. In some embodiments, the alkylamine is C4-6alkylamine. In some embodiments, the alkylamine is C6-8alkylamine. In some embodiments, the alkylamine is C8-10alkylamine. In some embodiments, the alkylamine is C10-12alkylamine. In some embodiments, the alkylamine is C12-14alkylamine. In some embodiments, the alkylamine is C14-18alkylamine. In some embodiments, the alkylamine is C18-20alkylamine. In some embodiments, the alkylamine is C10-18alkylamine. In some embodiments, the alkylamine is C22-24alkylamine. In some embodiments, the alkylamine is C24-30alkylamine. In some embodiments, the alkylamine is a straight chain alkylamine.

In various embodiments, the amphiphilic polymer is selected from a C2, C3, C4, C6, C7, C8, C9, C10, C11, C12, C13, C14, C15, C16, C17, C18, C19, C20, C21, C22, C23, C24, C25, C26, C27, C28, C29, C30, C31, C32, C34, C35, C36, C37, C38, C39 and C40 straight chain alkylamine.

In various embodiments, the nanospheres used in the present invention comprise a hydrophobic NSAID derivative, tocopherol and a therapeutic agent or a diagnostic agent conjugated to a hydrophilic, hydrophobic, or amphiphilic spacer.

In certain embodiments, the nanospheres comprise a hydrophobic NSAID derivative, tocopherol and an antioxidant α-lipoic acid-containing hydrophobic compound and therapeutic agent or a diagnostic agent conjugated to a hydrophilic, hydrophobic, or amphiphilic spacer.

In certain embodiments, the nanospheres comprise tocopherol and a hydrophobic antioxidant and anti-inflammatory derivative of an NSAID and a therapeutic agent or a diagnostic agent conjugated to a hydrophilic, hydrophobic, or amphiphilic spacer.

In certain embodiments, the nanospheres comprise tocopherol and derivatives of statin lactones and a therapeutic agent or a diagnostic agent conjugated to an amphiphilic spacer.

In various embodiments, the nanospheres comprise a hydrophobic NSAID derivative, tocopherol and a therapeutic agent or a diagnostic agent conjugated to an amphiphilic polymer.

In certain embodiments, the nanospheres comprise a hydrophobic NSAID derivative, tocopherol and an antioxidant α-lipoic acid-containing hydrophobic compound and therapeutic agent or a diagnostic agent conjugated to an amphiphilic polymer.

In certain embodiments, the nanospheres comprise tocopherol and a hydrophobic antioxidant and anti-inflammatory derivative of an NSAID and a therapeutic agent or a diagnostic agent conjugated to an amphiphilic polymer.

In certain embodiments, the nanospheres comprise tocopherol and derivatives of statin lactones and a therapeutic agent or a diagnostic agent conjugated to an amphiphilic polymer.

### Labeling

Various embodiments provide for methods of imaging and diagnosing TBI. The method can comprise providing a TBI-targeted nanosphere of the present invention, wherein the nanosphere further comprises a detectable label; administering the nanosphere to a subject in need thereof; and imaging the subject to detect the TBI. In some embodiments, detectable label is conjugated to a hydrophilic spacer, a hydrophobic spacer, an amphiphilic spacer, or an amphiphilic polymer.

As used herein, the term “detectable label” refers to a composition capable of producing a detectable signal indicative of the presence of a target. Generally, a detectable label is any composition detectable by spectroscopic, photochemical, biochemical, immunochemical, electrical, optical or chemical means. Suitable labels include fluorescent molecules, radioisotopes, nucleotide chromophores, enzymes, substrates, chemiluminescent moieties, magnetic particles, bioluminescent moieties, and the like.

In some embodiments, the detectable label can be an imaging agent, diagnostic agent, or contrast agent. As used herein, the term “imaging agent” refers to an element or functional group in a molecule that allows for the detection, imaging, and/or monitoring of the presence and/or progression of a condition(s), pathological disorder(s), and/or disease(s). The imaging agent can be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber. As used herein the term “contrast agent” refers to any molecule that changes the optical properties of tissue or organ containing the molecule. Optical properties that can be changed include, but are not limited to, absorbance, reflectance, fluorescence, birefringence, optical scattering and the like. In some embodiments, the detectable labels also encompass any imaging agent (e.g., but not limited to, a bubble, a liposome, a sphere, a contrast agent, or any detectable label described herein) that can facilitate imaging or visualization of a tissue or an organ in a subject, e.g., for diagnosis of an infection. In some embodiments, the imaging agent can be an antibody, or an epitope binding fragment thereof, that binds a protein expressed or overexpressed in TBI.

Suitable optical reporters include, but are not limited to, fluorescent reporters and chemiluminescent groups. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

Exemplary fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Many suitable forms of these fluorescent compounds are available and can be used.

Other exemplary detectable labels include luminescent and bioluminescent markers (e.g., biotin, luciferase (e.g., bacterial, firefly, click beetle and the like), luciferin, and aequorin), radiolabels (e.g., 3H, 125I, 35S, 14C, or 32P), enzymes (e.g., galactosidases, glucorinidases, phosphatases (e.g., alkaline phosphatase), peroxidases (e.g., horseradish peroxidase), and cholinesterases), and calorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference.

Suitable echogenic gases include, but are not limited to, a sulfur hexafluoride or perfluorocarbon gas, such as perfluoromethane, perfluoroethane, perfluoropropane, perfluorobutane, perfluorocyclobutane, perfluropentane, or perfluorohexane. Suitable non-metallic isotopes include, but are not limited to, ¹¹C, ¹⁴C, ¹³N, ¹⁸F, ¹²³I, ¹²⁴I, and ¹²⁵I. Suitable radioisotopes include, but are not limited to, ⁹⁹mTc, ⁹⁵Tc, ¹¹¹In, ⁶²Cu, ⁶⁴Cu, Ga, ⁶⁸Ga, and ¹⁵³Gd. Suitable paramagnetic metal ions include, but are not limited to, Gd(III), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir.

In some embodiments, the radionuclide can be bound to a chelating agent. Suitable radionuclides for direct conjugation include, without limitation, ¹⁸F, ¹²⁴I, ¹²⁵I, ¹³¹I, and mixtures thereof. Suitable radionuclides for use with a chelating agent include, without limitation, ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Suitable chelating agents include, but are not limited to, DOTA, BAD, TETA, DTPA, EDTA, NTA, HDTA, their phosphonate analogs, and mixtures thereof.

Means of detecting such labels are well known to those of skill in the art. Thus, for example, radiolabels can be detected using photographic film or scintillation counters, fluorescent markers can be detected using a photo-detector to detect emitted light. Enzymatic labels are typically detected by providing the enzyme with an enzyme substrate and detecting the reaction product produced by the action of the enzyme on the enzyme substrate, and calorimetric labels can be detected by visualizing the colored label. Exemplary methods for in vivo detection or imaging of detectable labels include, but are not limited to, radiography, magnetic resonance imaging (MRI), Positron emission tomography (PET), Single-photon emission computed tomography (SPECT, or less commonly, SPET), Scintigraphy, ultrasound, CAT scan, photoacoustic imaging, thermography, linear tomography, poly tomography, zonography, orthopantomography (OPT or OPG), and computed Tomography (CT) or Computed Axial Tomography (CAT scan).

In some embodiments, the detectable label is a fluorophore or a quantum dot. Without wishing to be bound by a theory, using a fluorescent reagent can reduce signal-to-noise in the imaging/readout, thus maintaining sensitivity.

In various embodiments, the imaging and/or diagnostic agents can include, but are not limited to fluorescent dyes, radiolabels, and antibodies against proteins overexpressed in TBI.

Exemplary fluorescent labeling reagents include, but are not limited to, Hydroxycoumarin, Succinimidyl ester, Aminocoumarin, Methoxycoumarin, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5 or Cy7.

Examples of radiolabels include but are not limited to ²H, ¹³C, ¹⁵N, iodophenylalanine, Tc99m, iodination.

One aspect of the invention relates to the fluorescent labeling of the nanospheres comprising NSAIDs. In some embodiments, a compound of Formula B-I-B-III is combined with an antioxidant and a thiol. In some embodiments the thiol is a C18-C20 thiol. In some embodiments the thiol is C18-C22 thiol. In some embodiments, the thiol is C18-C25 thiol. In some embodiments, the thiol is C18-C28 thiol. In some embodiments, the thiol is C18-C32 thiol. In some embodiments, the thiol is C20-C25 thiol.

In some embodiments the compound of Formula B-I-B-III is combined with an antioxidant and a thiol, and further with a fluorescent tag. In some embodiments, the fluorescent tag comprises a maleimide functionality. In some embodiments, the fluorescent tag is a cyanine. In some embodiments, the cyanine is Cy3 or Cy5. In some embodiments, the fluorescent tag is an Alexa fluor dye, FluoProbes dye, Sulfo Cy dye or Seta dye. In some embodiments, the fluorescent is another fluorophore.

Methods of Using the Nanospheres

Additional embodiments of the present invention provide for methods of using the NSAID nanospheres of the present invention, the antioxidant and NSAID nanospheres of the present invention, the NSAID nanosphere/antioxidant nanosphere composition of the present invention, or NSAID/antioxidant nanosphere combination of the present invention.

In some embodiments, methods of delivering a nanosphere to a traumatic brain injury (TBI) tissue in a subject, comprising: administering a therapeutically effective amount of a nanosphere to the subject, wherein the nanosphere comprises a compound selected from Formula B-I, Formula B-II, Formula B-III, and any combinations thereof.

In some embodiments, the nanosphere further comprises an antioxidant. In certain embodiments, the antioxidant is tocopherol or a derivative thereof.

In some embodiments, the nanosphere further comprises a compound of Formula A-IV or Formula A-V.

In some embodiments, the nanosphere further comprises an amphiphilic spacer.

In some embodiments, the amphiphilic spacer comprises a chemically active group selected from the group consisting of thiol, amine, carboxylic acid, carboxylic acid NHS ester, maleimide, hydrazine, ketone, aledehyde, and combinations thereof.

In some embodiments, the amphiphilic spacer is an alkylthiol or an alkylamine. In some embodiments, the amphiphilic spacer is 1-octadecanethiol.

In some embodiments, the nanosphere further comprises a polymer. In some embodiments, the polymer is poly(lactide-co-glycolide) (PLGA).

In some embodiments, the nanosphere further comprises a therapeutic agent.

In some embodiments, the therapeutic agent is selected from the group consisting of: a statin, nonsteroidal anti-inflammatory drug (NSAID), erythropoietin, peptide, antisense nucleic acid, DNA, RNA, protein, and combinations thereof.

In some embodiments, the therapeutic agent is delivered to the TBI tissue.

In some embodiments, the delivery of the therapeutic agent to the TBI tissue treats TBI.

In some embodiments, the therapeutic agent is conjugated to a hydrophilic spacer, a hydrophobic spacer, an amphiphilic spacer, or an amphiphilic polymer.

In some embodiments, the nanosphere further comprises an imaging agent. In some embodiments, the imaging agent is conjugated to a hydrophilic spacer, a hydrophobic spacer, an amphiphilic spacer, or an amphiphilic polymer.

In some embodiments, the imaging agent is selected from the group consisting of fluorescent molecules, radioisotopes, nucleotide chromophores, enzymes, substrates, chemiluminescent moieties, magnetic particles, bioluminescent moieties, antibody against a protein expressed or overexpressed in TBI, and combinations thereof.

In some embodiments, delivering the nanosphere to the TBI tissue treats TBI.

In some embodiments nanospheres can be used for treating inflammation or diseases or disease conditions that are caused by or related to inflammation in subjects in need thereof. The method comprises providing a composition comprising the NSAID nanospheres of the present invention, the antioxidant and NSAID nanospheres of the present invention, the NSAID nanosphere/antioxidant nanosphere composition of the present invention, or NSAID/antioxidant nanosphere combination of the present invention, and administering a therapeutically effective amount of the composition to the subject in need thereof.

In some particular embodiments, the NSAID nanospheres of the present invention, the antioxidant and NSAID nanospheres of the present invention, the NSAID nanosphere/antioxidant nanosphere composition of the present invention, or NSAID/antioxidant nanosphere combination of the present invention are used to treat traumatic brain injury (TBI) in a subject in need thereof. The method comprises providing a composition comprising the NSAID nanospheres of the present invention, the antioxidant and NSAID nanospheres of the present invention, the NSAID nanosphere/antioxidant nanosphere composition of the present invention, or NSAID/antioxidant nanosphere combination of the present invention and administering a therapeutically effective amount of the composition to the subject.

In some particular embodiments, the nanospheres comprising NSAID of the present are used to treat traumatic brain injury (TBI) in a subject in need thereof. The method comprises providing a composition comprising nanospheres comprising NSAID of the present invention, and administering a therapeutically effective amount of the composition to the subject.

In some embodiments, the method of treating TBI comprises providing a nanosphere of the present invention wherein the nanosphere further comprises a therapeutic agent conjugated to a hydrophilic spacer, a hydrophobic spacer, an amphiphilic spacer, or an amphiphilic polymer; and administering the nanosphere to a subject in need thereof.

In some embodiments, the method of treating TBI comprises providing a nanosphere of the present invention wherein a therapeutic agent is not conjugated to a hydrophilic spacer, a hydrophobic spacer, an amphiphilic spacer, or an amphiphilic polymer; and administering the nanosphere to a subject in need thereof.

In another embodiment, the NSAID nanospheres of the present invention, the antioxidant and NSAID nanospheres of the present invention, the NSAID nanosphere/antioxidant nanosphere composition of the present invention, or NSAID/antioxidant nanosphere combination of the present invention can be used as a carrier of a therapeutic agent. In one embodiment, the therapeutic agent is an additional NSAID that is useful for TBI treatment. In another embodiment, the therapeutic agent is an additional agent that is useful for TBI treatment. Accordingly, the present invention provides for a composition comprising the NSAID nanospheres of the present invention, the antioxidant and NSAID nanospheres of the present invention, the NSAID nanosphere/antioxidant nanosphere composition of the present invention, or NSAID/antioxidant nanosphere combination of the present invention and a therapeutic agent.

In another embodiment, the NSAID nanospheres of the present invention, the antioxidant and NSAID nanospheres of the present invention, the NSAID nanosphere/antioxidant nanosphere composition of the present invention, or NSAID/antioxidant nanosphere combination of the present invention can also be used as pharmaceutical and/or drug delivery vehicles to deliver small molecules, peptides, oligonucleotides, polynucleotides, proteins, antigens, chemotherapeutics, antisense nucleic acid molecules and the like, to tissue, organ, cell, etc.

### Methods of Preparing the Nanospheres

In another embodiment, the present invention provides for a method of preparing NSAID nanospheres comprising an NSAID derivative of the present invention. The method comprises providing an NSAID derivative of formula B-I and processing the NSAID derivative in a spontaneous emulsification process.

In another embodiment, the present invention provides for a method of preparing the NSAID/antioxidant nanosphere combination of the present invention. The antioxidant nanosphere can be a molecule as described by International Application No. PCT/US08/88541, which is incorporated herein by reference in its entirety as though fully set forth (e.g., formulas A-IV and A-V). The method comprises providing an NSAID derivative of formula B-I and an antioxidant molecule of formula A-IV or A-V and processing the NSAID derivative and antioxidant molecule in a spontaneous emulsification process. In another embodiment the method comprises providing molecules of Formula B-II and/or Formula B-III and an antioxidant molecule of formula A-IV or A-V and processing the molecules of Formula B-II and/or Formula B-III and antioxidant molecule in a spontaneous emulsification process.

In another embodiment, the present invention provides for a method of preparing the antioxidant and NSAID nanospheres. The method comprises providing a molecule of formula B-II or formula B-III and processing the molecule in a spontaneous emulsification process.

### Pharmaceutical Compositions

In various embodiments, the present invention provides pharmaceutical compositions including a pharmaceutically acceptable excipient along with a therapeutically effective amount of the nanospheres of the present invention. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

In various embodiments, the pharmaceutical compositions according to the invention can be formulated for delivery via any route of administration. “Route of administration” can refer to any administration pathway known in the art, including but not limited to aerosol, nasal, oral, transmucosal, transdermal, parenteral, enteral, or ocular. “Transdermal” administration can be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions can be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions can be in the form of solutions or suspensions for infusion or for injection. Via the topical route, the pharmaceutical compositions based on compounds according to the invention can be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they can be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier can be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it can come in contact, meaning that it must not carry a risk of toxicity, irritation, allergic response, immunogenicity, or any other complication that excessively outweighs its therapeutic benefits.

Pharmaceutically acceptable carriers include any and all solvents, dispersion media, coatings, antibacterial and antifungal agents, isotonic and absorption delaying agents, and the like which are compatible with the activity of the active agent and are physiologically acceptable to the subject. Some examples of materials which can serve as pharmaceutically-acceptable carriers include: (i) sugars, such as lactose, glucose and sucrose; (ii) starches, such as corn starch and potato starch; (iii) cellulose, and its derivatives, such as sodium carboxymethyl cellulose, methylcellulose, ethyl cellulose, microcrystalline cellulose and cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, corn oil and soybean oil; (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C2-C12 alcohols, such as ethanol; and (xxv) other non-toxic compatible substances employed in pharmaceutical formulations.

The pharmaceutical compositions according to the invention can also be encapsulated, tableted or prepared in an emulsion or syrup for oral administration. Pharmaceutically acceptable solid or liquid carriers can be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier can also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation can be administered directly p.o. or filled into a soft gelatin capsule.

The pharmaceutical compositions according to the invention can be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### Dosage

Typical dosages of an effective amount of the antioxidant derivatives of the composition of the invention can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described.

### Kits

The present invention is also directed to a kit to delivery a nanosphere of the present invention to TBI tissue and a kit to treat TBI. The kit is an assemblage of materials or components, including at least one of the inventive compositions. Thus, in some embodiments the kit contains a composition including the nanospheres of the present invention as described above.

The exact nature of the components configured in the inventive kit depends on its intended purpose. For example, some embodiments are configured for the purpose of delivering a nanosphere of the present invention to TBI tissue, and other embodiments are configured for the purpose of treating TBI. In some embodiments, the kit is configured particularly for the purpose of delivering to or treating mammalian subjects. In another embodiment, the kit is configured particularly for the purpose of delivering to or treating human subjects. In further embodiments, the kit is configured for veterinary applications, treating subjects such as, but not limited to, farm animals, domestic animals, and laboratory animals. In other embodiments, the kit is configured particularly for diagnostic purposes; for example, diagnosing TBI.

Instructions for use can be included in the kit. “Instructions for use” typically include a tangible expression describing the technique to be employed in using the components of the kit to effect a desired outcome, such as to delivery a nanosphere of the present invention to TBI tissue or to treat TBI. Optionally, the kit also contains other useful components, such as, diluents, buffers, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of inventive nanospheres comprising a therapeutic agent or an imaging agent optionally conjugated to a hydrophilic spacer, a hydrophobic spacer, an amphiphilic spacer, or an amphiphilic polymer. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1

Ethics Statement:

All experiments were approved by the Institutional Animal Care and Use Committee (IACUC) at the Cedars-Sinai Medical Center (protocol #2620). All efforts were made to minimize suffering through the use of anesthesia, analgesia, and post-injury care and monitoring.

Animals:

Male 12-week old C57BL/6 wild type mice (strain #000664) were obtained from Jackson Laboratory (Bar Harbor, Me.). On the day of injury, the placebo group and the treatment group had similar weights (26.01±1.18 g v, 25.48±1.64 g, p=0.42). Mice were anesthetized with inhalation isoflurane (4% to induction, and 2% maintenance), shaved in the region of cortical impact, and secured in a stereotaxic frame. Mice were then subjected to TBI using electromagnetic controlled cortical impact (CCI) [22,60]. A 2 mm impactor tip struck the left frontotemporal skull at a velocity of 3 m/s reaching a depth of 2 mm. One mouse assigned to the saline group died on impact, and one mouse each in the IP group and IV group were sacrificed before testing due to a failure to recover a righting reflex as a result of the injury. This reflects an expected 10% attrition due to the severity of the injury delivered.

Eight mice (n=8) were randomized to placebo group, undergoing CCI and then an intraperitoneal injection of phosphate buffered saline (PBS) after injury. Twelve mice (n=12) were assigned to the intraperitoneal (IP) treatment group and were given an immediate IP injection of nanoprodrug (100 μl, 0.2 mg/mouse). Six mice (n=6) were assigned to the intravenous (IV) group, recovered from anesthesia for five minutes, and injected with nanoprodrug (100 μl, 0.2 mg/mouse) via tail vein. All mice were recovered on a warming pad until ambulatory, returned to their cages, and housed in groups of two with a 14:10 hour light-dark cycle with water and softened chow available ad libitum.

Preparation of NSAID Nanoprodrug:

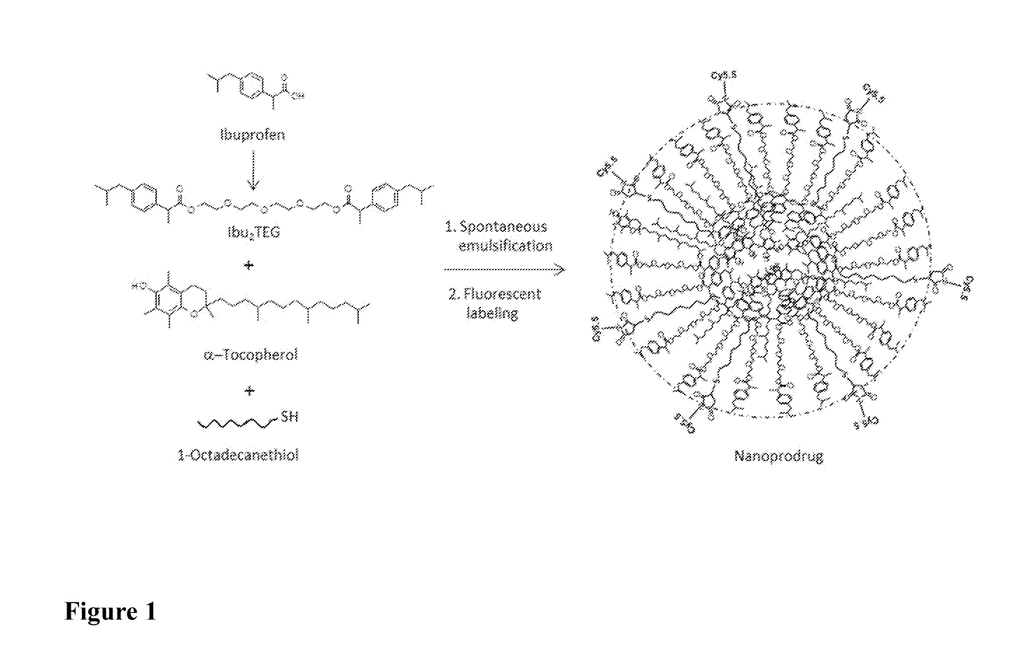

The nanoprodrug is constructed of ibuprofen molecules joined by a tetra ethylene glycol (TEG) spacer in an emulsion with the antioxidant α-tocopherol and 1-octadecanethiol which is irreversibly bonded to the Cy5.5 fluorescent tracer. The combination of two ibuprofen molecules joined by the TEG spacer is referred to as Ibu₂TEG (FIG. 1). The ester bond between each ibuprofen and the TEG spacer is biodegradable, ensuring that the prodrug molecules break down hydrolytically or enzymatically. In contrast, the thioether bond between the Cy5.5 maleimide fluorescent tracer and 1-octadecanethiol is not biodegradable. 1-Octadecanethiol is a water-insoluble sulfur compound with an 18 carbon alkyl chain, which forms a strong hydrophobic assembly with Ibu₂TEG and α-tocopherol. The nanoprodrug was noted to be highly stable. We incubated the complete nanoprodrug for 48 hours at physiological pH in PBS and did not detect any detachment of Cy5.5 from the nanoprodrug particles (data not shown).

The details of Ibu₂TEG synthesis are previously described [61]. The nanoprodrug was prepared by the spontaneous emulsification of 50 mg of Ibu₂TEG, 10 mg α-tocopherol, and 2 mg of 1-octadecanethiol all dissolved in acetone (5 ml) containing polysorbate 80 (0.1% w/v). The organic solution was poured under moderate stirring on a magnetic plate into an aqueous phase prepared by dissolving 25 mg of Pluronic F68 in 10 ml distilled water (0.25% w/v). Following 15 min of stirring, acetone was removed under reduced pressure. To 2 mL of the thiolated nanoprodrug suspension, 500 μL of 10×PBS and molar equivalent of Cy5.5 maleimide (GE Healthcare) were added. The reaction mixture was incubated overnight at room temperature under light protection. To remove unbound Cy5.5 maleimide, the suspension was purified on a G-25 Sephadex column (GE Healthcare) equilibrated with 20 mM sodium citrate buffer with 0.15 M NaCl. The suspension was dialyzed in a cellulose membrane tube (Sigma #D9777) overnight in distilled water and filtered consecutively through 0.8, 0.45, and 0.2 μm hydrophilic syringe filters (Corning) and stored at 4° C. The concentration of the bound Cy5.5 was determined by mixing 200 μL of nanoprodrug suspension with 800 μL acetonitrile before measuring the optical light absorbance at 675 nm. The concentration was calculated using a standard curve generated with Cy5.5 maleimide.

Behavioral Testing:

Behavioral testing was determined using the Barnes Maze for cognitive function, and the open field and rotorod tests of motor function. The Barnes Maze assessed spatial reference and working memory retention. Ten animals were tested in the Barnes Maze (n=6 IP, n=4 placebo). Prior to injury animals received five days of training to locate and enter a hide box within a two-minute time limit. Injury occurred on day 6, and memory retention of the task was assessed on day 7. On day 8, a probe test was conducted as a control, in which the box was moved to a new location to determine if the animals were not using non-memory associated cues (such as the scent of the box) to locate the hide box.

The open field and rotorod tests were conducted twenty-four hours after TBI to assess gross motor function. In the open field test, mice were placed in a plexiglass box, with motion monitored by lasers over the course of one hour. Ambulation is defined as more than two consecutive laser beam breaks. The rotorod test assesses coordination and strength by measuring the time the animal can balance on a rod rotating at constantly increasing angular velocity.

Fluorescence Imaging:

Post mortem brain tissue was imaged using Xenogen 200 Imaging System (Caliper Life Sciences) to localize accumulation of the fluorescent nanoprodrug within the brain. Intact whole brains were imaged and then sectioned for repeat imaging. Frozen tissue was mounted in OCT compound, cryosectioned using a cryotomb (10 μm), and stained with hematoxylin and eosin. For fluorescent confocal microscopy, brains were cryosectioned (10 μm) and mounted and coverslipped with one drop of mounting medium with DAPI (Prolong Gold, Invitrogen). A fluorescent microscope (Model Upright Zeiss) and a confocal laser-scanning microscope (Leica Microsystem SP5) equipped with a digital camera were used for microscopic analysis.

Tissue Collection:

After behavioral testing and imaging procedures, mice were sacrificed three days after injury using carbon dioxide inhalation followed by cervical dislocation. Brains were then immediately harvested by peeling the skull away and extracting the whole brain onto dry ice for snap freezing. Tissues were stored at −80 degrees Celsius until processing.

Statistics:

Groups are described as means with standard deviations and compared using a two tailed Student's t-test, with a level of p=0.05 considered significant.

### Results

Imaging:

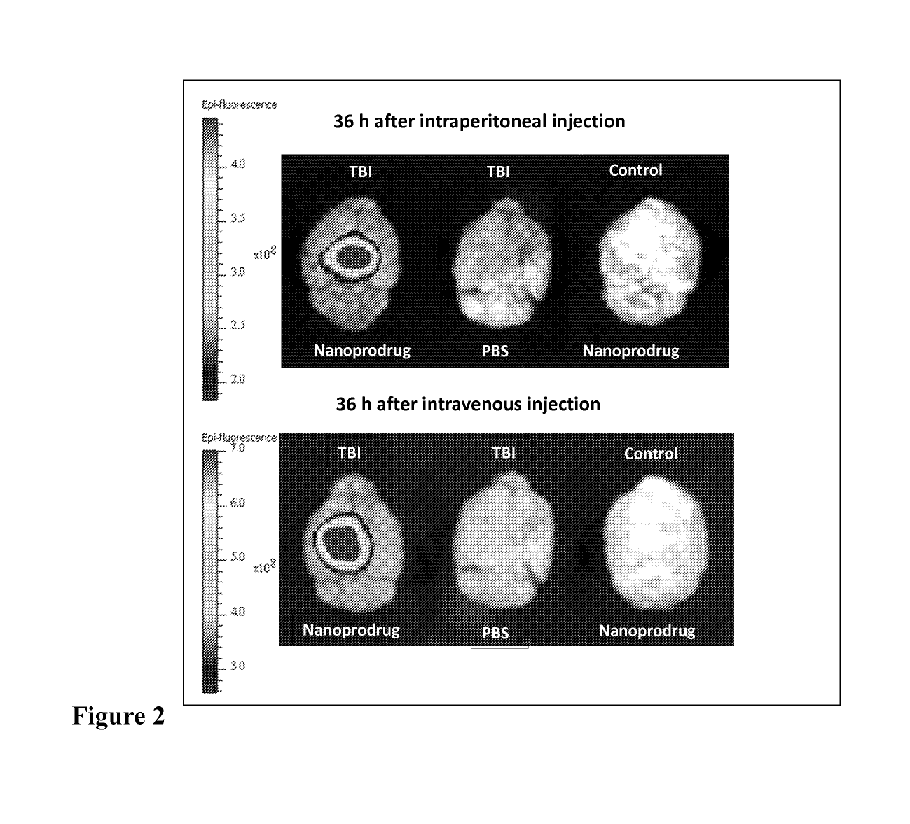

Whole brains were collected from mice 36 hours after injury and nanoprodrug administration. Using Xenogen bioluminescence imaging, the Cy5.5 fluorescent marker was detected at the site of injury on the left parietal region of whole brain (FIG. 2). Fluorescence was not detected in uninjured sham animals receiving the nanoprodrug, nor was it detected in TBI animals treated with PBS. Comparing animals treated with IP injection of the drug (FIG. 2, upper panel) to animals treated with IV injection of the drug (lower panel), accumulation is similar.

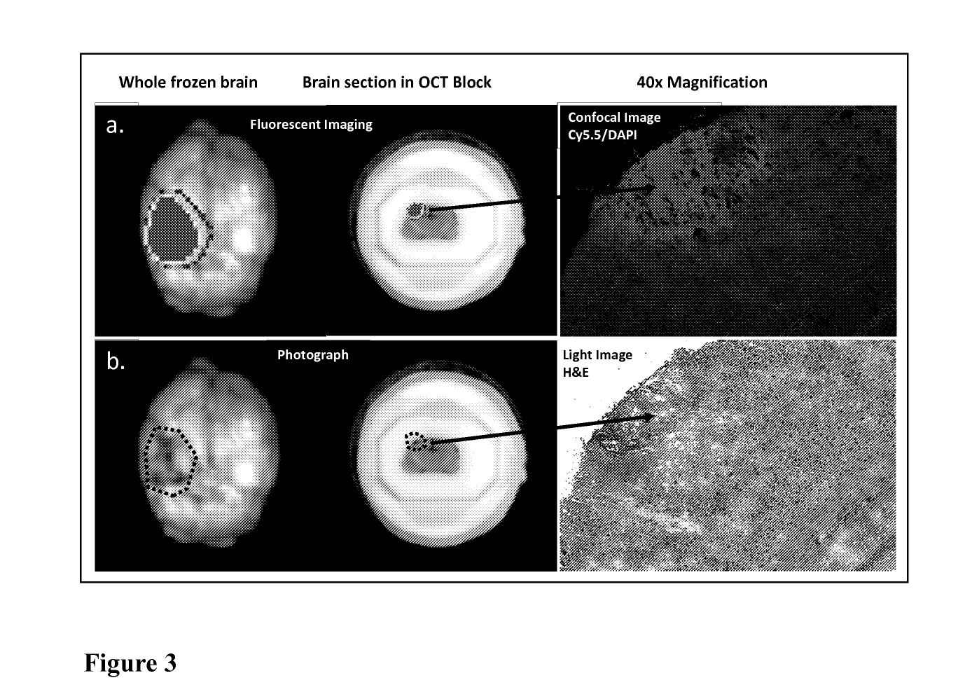

Confocal microscopy of sectioned brains revealed accumulation of the nanoprodrug (pink, Cy5.5) at the area of injury (FIG. 3, right upper panel). Nuclei are stained with DAPI nuclear stain in blue. Whole brain photographs exhibit hematoma formation and hematoxylin and eosin stain of brain tissue slices demonstrates significant tissue disruption of the cortical tissue (FIG. 3, lower right panel).

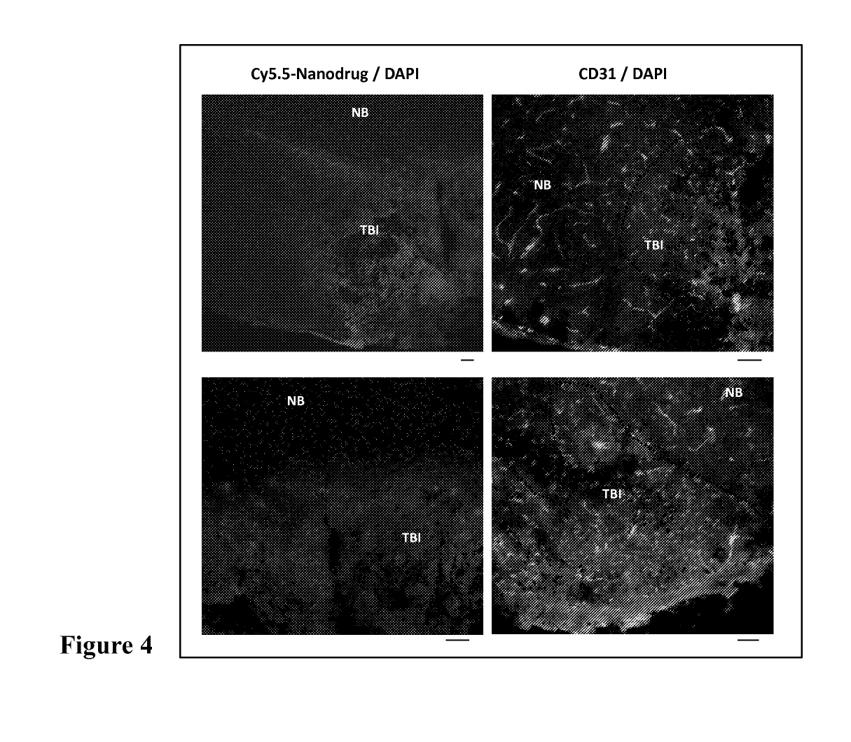

To investigate the cerebral vasculature, we stained for CD31+ vascular endothelial cells (FIG. 4). The tubular structures of vessels are visible outside of the focal region of impact. In contrast, vessel organization is highly disorganized at the area of TBI. This disorganization is highly correlated with regions of increased nanoprodrug uptake.

Behavior:

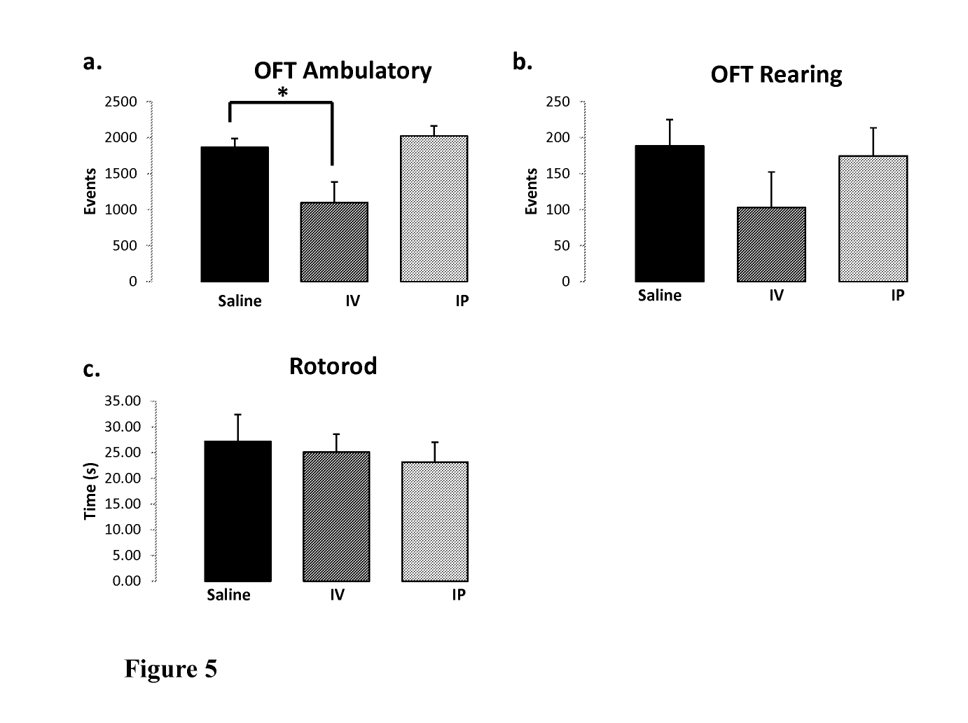

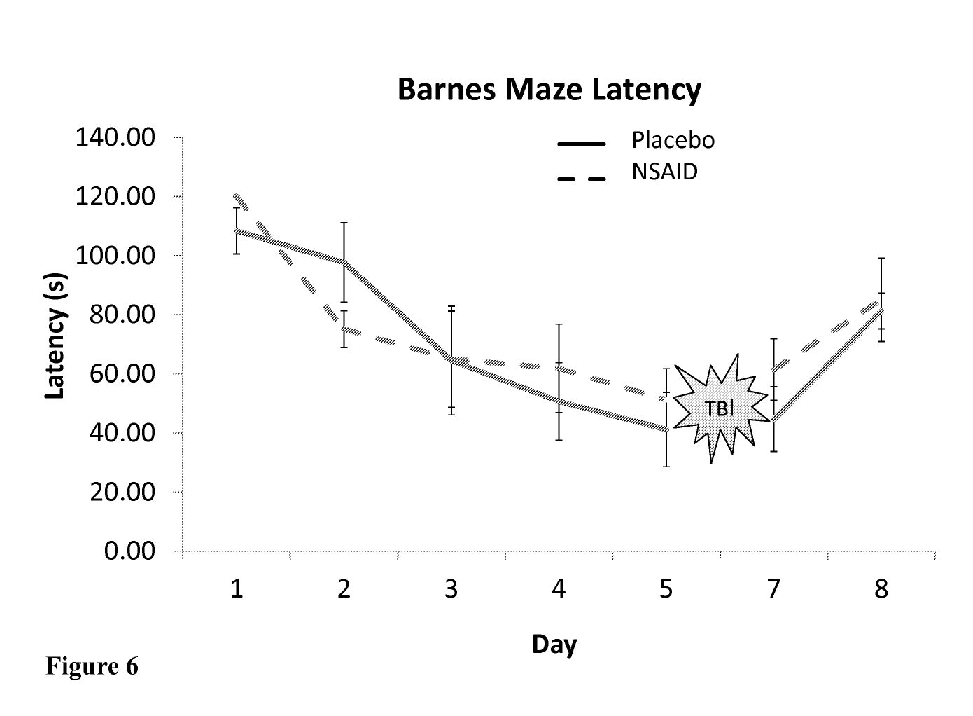

On the open field test, mice treated with the nanoprodrug IP had no significant reduction in ambulation events compared to placebo (nanoprodrug IP 2024±484 v. placebo 1865±302, p=0.47) or rearing (nanoprodrug IP 174±136 v. placebo 188±90, p=0.82) (FIG. 5). In contrast, mice treated with the nanoprodrug IV had significantly reduced ambulation compared to the control group (nanoprodrug IV 1098±641 v. NS 1865±302, p=0.005). No difference in rearing events were noted with IV injection of the prodrug (nanoprodrug IV 103±110.6 v. placebo 188±89.5, p=0.31). No significant differences were noted in rotorod times between groups (nanoprodrug IV 25.1±7.7 s v. placebo 27.2±12.8 s, p=0.76; nanoprodrug IP 23.2±13.4 s v. placebo 27.2±12.8 s, p=0.55). No significant differences were noted in latency to find the hide box between groups on post injury day 1 (IP 61.4±10.4 s vs placebo 44.67±11.0 s p=0.32) (FIG. 6). No significant differences were noted between groups on the reversal test (IP 85.06±14.1 v placebo 81.25±6.0 p=0.84).

### REFERENCES

1. Rutland-Brown W, Langlois J A, Thomas K E, Xi Y L (2006) Incidence of traumatic brain injury in the United States, 2003. The Journal of Head Trauma Rehabilitation 21: 544-548.
2. Summers C R, Ivins B, Schwab K A (2009) Traumatic Brain Injury in the United States: An Epidemiologic Overview. Mount Sinai Journal of Medicine 76: 105-110.
3. Xu J, Kochanek K D, Murphy S L, Tejada-Vera B, others (n.d.) Deaths: final data for 2007. National Vital Statistics Reports 58: 19.
4. WISQARS Years of Potential Life Lost (YPLL) Reports, 1999-2007 (n.d.). National Center for Injury Prevention and Control. Available: http://webappa.cdc.gov/sasweb/ncipc/ypll10.html.
5. Faul M, Xu L, Wald M M, Coronado V G (2010) Traumatic brain injury in the United States: emergency department visits, hospitalizations, and deaths. Atlanta (Ga.): Centers for Disease Control and Prevention, National Center for Injury Prevention and Control.
6. Thurman D J (2001) Epidemiology and economics of head trauma. Head trauma: basic, preclinical, and clinical directions. New York: Wiley and Sons. pp. 1193-1202.
7. Marik P E, Varon J, Trask T (2002) Management of Head Trauma. Chest 122: 699-711.
8. Goldman S M, Tanner C M, Oakes D, Bhudhikanok G S, Gupta A, et al. (2006) Head injury and Parkinson's disease risk in twins. Annals of Neurology 60: 65-72. doi:10.1002/ana.20882.
9. Lye T C, Shores E A (2000) Traumatic brain injury as a risk factor for Alzheimer's disease: a review. Neuropsychology Review 10: 115-129.
10. Muehlschlegel S, Sims J R (2009) Dantrolene: mechanisms of neuroprotection and possible clinical applications in the neurointensive care unit. Neurocrit Care 10: 103-115. doi:10.1007/s12028-008-9133-4.
11. Langham J, Goldfrad C, Teasdale G, Shaw D, Rowan K (2003) Calcium channel blockers for acute traumatic brain injury. In: The Cochrane Collaboration, Langham J, editors. Cochrane Database of Systematic Reviews. Chichester, U K: John Wiley & Sons, Ltd. Available: http://onlinelibrary.wiley.com/o/cochrane/clsysrev/articles/CD000565/abstract.html. Accessed 27 May 2011.
12. Ravin R, Blank P S, Steinkamp A, Rappaport S M, Ravin N, et al. (2012) Shear forces during blast, not abrupt changes in pressure alone, generate calcium activity in human brain cells. PLoS ONE 7: e39421. doi:10.1371/journal.pone.0039421.
13. Maas A I R, Murray G, Henney H 3rd, Kassem N, Legrand V, et al. (2006) Efficacy and safety of dexanabinol in severe traumatic brain injury: results of a phase III randomised, placebo-controlled, clinical trial. Lancet Neurol 5: 38-45. doi:10.1016/S1474-4422(05)70253-2.
14. Chung E S, Bok E, Chung Y C, Baik H H, Jin B K (2012) Cannabinoids prevent lipopolysaccharide-induced neurodegeneration in the rat substantia nigra in vivo through inhibition of microglial activation and NADPH oxidase. Brain Res 1451: 110-116. doi:10.1016/j.brainres.2012.02.058.
15. Homsi S, Federico F, Croci N, Palmier B, Plotkine M, et al. (2009) Minocycline effects on cerebral edema: Relations with inflammatory and oxidative stress markers following traumatic brain injury in mice. Brain research 1291: 122-132.
16. Viscomi M T, Latini L, Florenzano F, Bernardi G, Molinari M (2008) Minocycline Attenuates Microglial Activation but Fails to Mitigate Degeneration in Inferior Olive and Pontine Nuclei After Focal Cerebellar Lesion. Cerebellum 7: 401-405. doi:10.1007/s12311-008-0042-z.
17. Sen A P, Gulati A (2010) Use of magnesium in traumatic brain injury. Neurotherapeutics 7: 91-99. doi:10.1016/j.nurt.2009.10.014.
18. Afshari D, Moradian N, Rezaei M (2012) Evaluation of the intravenous magnesium sulfate effect in clinical improvement of patients with acute ischemic stroke. Clinical neurology and neurosurgery. Available: http://www.ncbi.nlm.nih.gov/pubmed/22749947. Accessed 27 Sep. 2012.
19. Vink R, Nimmo A J (2009) Multifunctional drugs for head injury. Neurotherapeutics 6: 28-42.
20. Narayan R K, Michel M E, Ansell B, Baethmann A, Biegon A, et al. (2002) Clinical trials in head injury. J Neurotrauma 19: 503-557. doi:10.1089/089771502753754037.
21. Jain K K (2008) Neuroprotection in traumatic brain injury. Drug Discov Today 13: 1082-1089. doi:10.1016/j.drudis.2008.09.006.
22. Yu S, Kaneko Y, Bae E, Stahl C E, Wang Y, et al. (2009) Severity of controlled cortical impact traumatic brain injury in rats and mice dictates degree of behavioral deficits. Brain Research 1287: 157-163. doi:10.1016/j.brainres.2009.06.067.
23. Lighthall J W (1988) Controlled Cortical Impact: A New Experimental Brain Injury Model. Journal of Neurotrauma 5: 1-15. doi:10.1089/neu.1988.5.1.
24. Lindgren S, Rinder L (1966) Experimental studies in head injury. Radiation and Environmental Biophysics 3: 174-180. doi:10.1007/BF01191611.
25. Feeney D M, Boyeson M G, Linn R T, Murray H M, Dail W G (1981) Responses to cortical injury: I. Methodology and local effects of contusions in the rat. Brain Research 211: 67-77. doi:10.1016/0006-8993(81)90067-6.
26. Long J B, Bentley T L, Wessner K A, Cerone C, Sweeney S, et al. (2009) Blast Overpressure in Rats: Recreating a Battlefield Injury in the Laboratory. Journal of Neurotrauma 26: 827-840. doi:10.1089/neu.2008.0748.
27. Koliatsos V E, Cernak I, Xu L, Song Y, Savonenko A, et al. (2011) A Mouse Model of Blast Injury to Brain. Journal of Neuropathology and Experimental Neurology 70: 399-416. doi:10.1097/NEN.0b013e3182189f06.
28. Fox G B, Fan L, Levasseur R A, Faden A I (1998) Sustained Sensory/Motor and Cognitive Deficits With Neuronal Apoptosis Following Controlled Cortical Impact Brain Injury in the Mouse. Journal of Neurotrauma 15: 599-614. doi:10.1089/neu.1998.15.599.
29. Acosta S A, Tajiri N, Shinozuka K, Ishikawa H, Grimmig B, et al. (2013) Long-Term Upregulation of Inflammation and Suppression of Cell Proliferation in the Brain of Adult Rats Exposed to Traumatic Brain Injury Using the Controlled Cortical Impact Model. PLoS ONE 8: e53376. doi:10.1371/journal.pone.0053376.
30. Terrio H, Brenner L A, Ivins B J, Cho J M, Helmick K, et al. (2009) Traumatic Brain Injury Screening. Journal of Head Trauma Rehabilitation 24: 14-23. doi:10.1097/HTR.0b013e31819581d8.
31. Okie S (2005) Traumatic brain injury in the war zone. N Engl J Med 352: 2043-2047. doi: 10.1056/NEJMp058102.
32. Hein A M, O'Banion M K (2009) Neuroinflammation and memory: the role of prostaglandins. Mol Neurobiol 40: 15-32. doi:10.1007/s12035-009-8066-z.
33. Wallenquist U, Holmqvist K, Hånell A, Marklund N, Hillered L, et al. (2012) Ibuprofen attenuates the inflammatory response and allows formation of migratory neuroblasts from grafted stem cells after traumatic brain injury. Restor Neurol Neurosci 30: 9-19. doi:10.3233/RNN-2011-0606.
34. Kovesdi E, Kamnaksh A, Wingo D, Ahmed F, Grunberg N E, et al. (2012) Acute minocycline treatment mitigates the symptoms of mild blast-induced traumatic brain injury. Front Neurol 3: 111. doi:10.3389/fneur.2012.00111.
35. Khaksari M, Soltani Z, Shahrokhi N, Moshtaghi G, Asadikaram G (2011) The role of estrogen and progesterone, administered alone and in combination, in modulating cytokine concentration following traumatic brain injury. Can J Physiol Pharmacol 89: 31-40. doi:10.1139/y10-103.
36. Uttara B, Singh A V, Zamboni P, Mahajan R T (2009) Oxidative stress and neurodegenerative diseases: a review of upstream and downstream antioxidant therapeutic options. Curr Neuropharmacol 7: 65-74. doi: 10.2174/157015909787602823.
37. Gilgun-Sherki Y, Melamed E, Offen D (2001) Oxidative stress induced-neurodegenerative diseases: the need for antioxidants that penetrate the blood brain barrier. Neuropharmacology 40: 959-975. doi:10.1016/S0028-3908(01)00019-3.
38. Morganti-Kossmann M C, Rancan M, Stahel P F, Kossmann T (2002) Inflammatory response in acute traumatic brain injury: a double-edged sword. Curr Opin Crit Care 8: 101-105.
39. Botteri M, Bandera E, Minelli C, Latronico N (2008) Cerebral blood flow thresholds for cerebral ischemia in traumatic brain injury. A systematic review. Crit Care Med 36: 3089-3092. doi:10.1097/CCM.0b013e31818bd7df.
40. Warden S J (2010) Prophylactic use of NSAIDs by athletes: a risk/benefit assessment. Phys Sportsmed 38: 132-138. doi:10.3810/psm.2010.04.1770.
41. Dannhardt G, Kiefer W (2001) Cyclooxygenase inhibitors—current status and future prospects. European Journal of Medicinal Chemistry 36: 109-126. doi:10.1016/S0223-5234(01)01197-7.
42. Tammara V K, Narurkar M M, Crider A M, Khan M A (1993) Synthesis and evaluation of morpholinoalkyl ester prodrugs of indomethacin and naproxen. Pharm Res 10: 1191-1199.
43. Bonina F P, Puglia C, Barbuzzi T, De Caprariis P, Palagiano F, et al. (2001) In vitro and in vivo evaluation of polyoxyethylene esters as dermal prodrugs of ketoprofen, naproxen and diclofenac. European Journal of Pharmaceutical Sciences 14: 123-134. doi: 10.1016/S0928-0987(01)00163-4.
44. Siskou I C, Rekka E A, Kourounakis A P, Chrysselis M C, Tsiakitzis K, et al. (2007) Design and study of some novel ibuprofen derivatives with potential nootropic and neuroprotective properties. Bioorg Med Chem 15: 951-961. doi: 10.1016/j.bmc.0.2006.10.056.
45. Burton G W, Joyce A, Ingold K U (1982) First proof that vitamin E is major lipid-soluble, chain-breaking antioxidant in human blood plasma. Lancet 2: 327.
46. Niki E, Noguchi N (2004) Dynamics of antioxidant action of vitamin E. Acc Chem Res 37: 45-51. doi:10.1021/ar030069m.
47. Halliwell B (2006) Oxidative stress and neurodegeneration: where are we now? J Neurochem 97: 1634-1658. doi:10.1111/j.1471-4159.2006.03907.x.
48. Breitner J C, Welsh K A, Helms M J, Gaskell P C, Gau B A, et al. (1995) Delayed onset of Alzheimer's disease with nonsteroidal anti-inflammatory and histamine H2 blocking drugs. Neurobiol Aging 16: 523-530.
49. Glass C K, Saijo K, Winner B, Marchetto M C, Gage F H (2010) Mechanisms Underlying Inflammation in Neurodegeneration. Cell 140: 918-934. doi:10.1016/j.cell.2010.02.016.
50. Bernardi A, Frozza R L, Meneghetti A, Hoppe J B, Battastini A M O, et al. (2012) Indomethacin-loaded lipid-core nanocapsules reduce the damage triggered by Aβ1-42 in Alzheimer's disease models. Int J Nanomedicine 7: 4927-4942. doi:10.2147/IJN.S35333.
51. Côté S, Carmichael P-H, Verreault R, Lindsay J, Lefebvre J, et al. (2012) Nonsteroidal anti-inflammatory drug use and the risk of cognitive impairment and Alzheimer's disease. Alzheimers Dement 8: 219-226. doi:10.1016/j.jalz.2011.03.012.
52. Chassidim Y, Veksler R, Lublinsky S, Pell G S, Friedman A, et al. (2013) Quantitative imaging assessment of blood-brain barrier permeability in humans. Fluids Barriers CNS 10: 9. doi:10.1186/2045-8118-10-9.
53. Stahel P F, Shohami E, Younis F M, Kariya K, Otto V I, et al. (2000) Experimental closed head injury: analysis of neurological outcome, blood-brain barrier dysfunction, intracranial neutrophil infiltration, and neuronal cell death in mice deficient in genes for pro-inflammatory cytokines J Cereb Blood Flow Metab 20: 369-380. doi:10.1097/00004647-200002000-00019.
54. Shlosberg D, Benifla M, Kaufer D, Friedman A (2010) Blood-brain barrier breakdown as a therapeutic target in traumatic brain injury. Nat Rev Neurol 6: 393-403. doi:10.1038/nrneurol.2010.74.
55. Prabhakar U, Blakey D C, Maeda H, Jain R K, Sevick-Muraca E M, et al. (2013) Challenges and key considerations of the enhanced permeability and retention effect (EPR) for nanomedicine drug delivery in oncology. Cancer Res. doi:10.1158/0008-5472.CAN-12-4561.
56. Maeda H (2001) The enhanced permeability and retention (EPR) effect in tumor vasculature: the key role of tumor-selective macromolecular drug targeting. Adv Enzyme Regul 41: 189-207.
57. Sangiorgi S, De Benedictis A, Protasoni M, Manelli A, Reguzzoni M, et al. (2013) Early-stage microvascular alterations of a new model of controlled cortical traumatic brain injury: 3D morphological analysis using scanning electron microscopy and corrosion casting. J Neurosurg. doi:10.3171/2012.11.JNS12627.
58. Pham L-D D, Hayakawa K, Seo J H, Nguyen M-N, Som A T, et al. (2012) Crosstalk between oligodendrocytes and cerebral endothelium contributes to vascular remodeling after white matter injury. Glia 60: 875-881. doi:10.1002/glia.22320.
59. Lin Y, Pan Y, Shi Y, Huang X, Jia N, et al. (2012) Delivery of large molecules via poly(butyl cyanoacrylate) nanoparticles into the injured rat brain. Nanotechnology 23: 165101. doi:10.1088/0957-4484/23/16/165101.
60. Brody D L, Mac Donald C, Kessens C C, Yuede C, Parsadanian M, et al. (2007)
Electromagnetic controlled cortical impact device for precise, graded experimental traumatic brain injury. J Neurotrauma 24: 657-673. doi:10.1089/neu.2006.0011.
61. Lee B-S, Yuan X, Xu Q, McLafferty F S, Petersen B A, et al. (2009) Stimuli-responsive antioxidant nanoprodrugs of NSAIDs. Int J Pharm 372: 112-124. doi:10.1016/j.ijpharm.2008.12.037.
62. Ziebell J M, Morganti-Kossmann M C (2010) Involvement of pro- and anti-inflammatory cytokines and chemokines in the pathophysiology of traumatic brain injury. Neurotherapeutics 7: 22-30. doi:10.1016/j.nurt.2009.10.016.
63. Tehranian R, Andell-Jonsson S, Beni S M, Yatsiv I, Shohami E, et al. (2002) Improved recovery and delayed cytokine induction after closed head injury in mice with central overexpression of the secreted isoform of the interleukin-1 receptor antagonist. J Neurotrauma 19: 939-951. doi:10.1089/089771502320317096.
64. Lu W Y, Rhoney D H, Boling W B, Johnson J D, Smith T C (1997) A Review of Stress Ulcer Prophylaxis in the Neurosurgical Intensive Care Unit. Neurosurgery 41: 416-426. doi: 10.1097/00006123-199708000-00017.
65. Lee B-S, Yoon C W, Osipov A, Moghavem N, Nwachokor D, et al. (2011) Nanoprodrugs of NSAIDs: Preparation and Characterization of Flufenamic Acid Nanoprodrugs. Journal of Drug Delivery 2011: 1-13. doi:10.1155/2011/980720.
66. Iadecola C, Gorelick P B (2005) The janus face of cyclooxygenase-2 in ischemic stroke shifting toward downstream targets. Stroke 36: 182-185.
67. Cernak I, O'Connor C, Vink R (2002) Inhibition of cyclooxygenase 2 by nimesulide improves cognitive outcome more than motor outcome following diffuse traumatic brain injury in rats. Experimental Brain Research 147: 193-199. doi:10.1007/s00221-002-1245-z.
68. Strauss K I, Barbe M F, Marshall R M, Raghupathi R, Mehta S, et al. (2000) Prolonged Cyclooxygenase-2 Induction in Neurons and Glia Following Traumatic Brain Injury in the Rat. Journal of neurotrauma 17: 695-711.
69. Fink K B, Andrews L J, Butler W E, Ona V O, Li M, et al. (1999) Reduction of post-traumatic brain injury and free radical production by inhibition of the caspase-1 cascade. Neuroscience 94: 1213-1218.
70. Liu M, Zhang Y, Zhang Y, Pei L, Liu X (2012) Prostaglandin E2 receptor 1 activity regulates cell survival following hypoxia in cultured rat cortical neurons. Neurosci Lett 508: 31-36. doi:10.1016/j.neulet.2011.12.010.
71. Nogawa S, Zhang F, Ross M E, Iadecola C (1997) Cyclo-oxygenase-2 gene expression in neurons contributes to ischemic brain damage. J Neurosci 17: 2746-2755.
72. Clifton G L, Robertson C S, Kyper K, Et al. (1983) Cardiovascular response to severe head injury. Journal of neurosurgery 59: 447-454.
73. Lee P-C, Bordelon Y, Bronstein J, Ritz B (2012) Traumatic brain injury, paraquat exposure, and their relationship to Parkinson disease. Neurology 79: 2061-2066. doi:10.1212/WNL0b013e3182749f28.
74. Goldman S M, Tanner C M, Oakes D, Bhudhikanok G S, Gupta A, et al. (2006) Head injury and Parkinson's disease risk in twins. Annals of Neurology 60: 65-72. doi:10.1002/ana.20882.
75. Rigotti A (2007) Absorption, transport, and tissue delivery of vitamin E. Mol Aspects Med 28: 423-436. doi:10.1016/j.mam.2007.01.002.
76. Uttara B, Singh A V, Zamboni P, Mahajan R T (2009) Oxidative stress and neurodegenerative diseases: a review of upstream and downstream antioxidant therapeutic options. Current neuropharmacology 7: 65-74.
77. Halliwell B (1989) Oxidants and the central nervous system: some fundamental questions. Is oxidant damage relevant to Parkinson's disease, Alzheimer's disease, traumatic injury or stroke? Acta Neurologica Scandinavica 80: 23-33. doi:10.1111/j.1600-0404.1989.tb01779.x.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A method of delivering a nanosphere to a traumatic brain injury (TBI) tissue in a subject, comprising:
administering a therapeutically effective amount of a nanosphere to the subject, wherein the nanosphere comprises a compound selected from Formula B-I, Formula B-II, Formula B-III, and any combinations thereof.

2. The method of claim 1, wherein the nanosphere further comprises an antioxidant.

3. The method of claim 1, wherein the antioxidant is tocopherol or a derivative thereof.

4. The method of claim 1, wherein the nanosphere further comprises a compound of Formula A-IV or Formula A-V.

5. The method of claim 1, wherein the nanosphere further comprises an amphiphilic spacer.

6. The method of claim 5, wherein the amphiphilic spacer comprises a chemically active group selected from the group consisting of thiol, amine, carboxylic acid, carboxylic acid NHS ester, maleimide, hydrazine, ketone, aledehyde, and combinations thereof, or the amphiphilic spacer is an alkylthiol or an alkylamine.

7. (canceled)

8. The method of claim 5, wherein the amphiphilic spacer is 1-octadecanethiol.

9. The method of claim 1, wherein the nanosphere further comprises a polymer.

10. The method of claim 9, wherein the polymer is poly(lactide-co-glycolide) (PLGA).

11. The method of claim 1, wherein the nanosphere further comprises a therapeutic agent.

12. The method of claim 11, wherein the therapeutic agent is selected from the group consisting of: a statin, nonsteroidal anti-inflammatory drug (NSAID), erythropoietin, peptide, antisense nucleic acid, DNA, RNA, protein, and combinations thereof.

13. The method of claim 12, wherein the therapeutic agent is delivered to the TBI tissue.

14. The method of claim 13, wherein the delivery of the therapeutic agent to the TBI tissue treats TBI.

15. The method of claim 11, wherein the therapeutic agent is conjugated to a hydrophilic spacer, a hydrophobic spacer, an amphiphilic spacer, or an amphiphilic polymer.

16. The method of claim 1, wherein the nanosphere further comprises an imaging agent.

17. The method of claim 16, wherein the imaging agent conjugated to a hydrophilic spacer, a hydrophobic spacer, an amphiphilic spacer, or an amphiphilic polymer.

18. (canceled)

19. The method of claim 1, wherein delivering the nanosphere to the TBI tissue treats TBI.

20. A method of detecting or diagnosing TBI in a subject in need thereof comprising:
administering an effective amount of a nanosphere to the subject, wherein the nanosphere comprises an imaging agent and a compound selected from Formula B-I, Formula B-II, Formula B-III, and any combinations thereof; and
imaging the subject to detect or diagnose the TBI.

21. The method of claim 20, wherein the imaging agent is conjugated to a hydrophilic spacer, a hydrophobic spacer, an amphiphilic spacer, or an amphiphilic polymer.

22. The method of claim 20, wherein the imaging agent is selected from the group consisting of fluorescent molecules, radioisotopes, nucleotide chromophores, enzymes, substrates, chemiluminescent moieties, magnetic particles, bioluminescent moieties, antibody against a protein expressed or overexpressed in TBI, and combinations thereof.

23. The method of claim 20, wherein the nanosphere further comprises an antioxidant.

24. The method of claim 23, wherein the antioxidant is tocopherol or a derivative thereof.

25. The method of claim 20, wherein the nanosphere further comprises a compound of Formula A-IV or Formula A-V.

26. The method of claim 20, wherein the nanosphere further comprises an amphiphilic spacer.

27. The method of claim 26, wherein the amphiphilic spacer comprises a chemically active group selected from the group consisting of thiol, amine, carboxylic acid, carboxylic acid NHS ester, maleimide, hydrazine, ketone, aledehyde, and combinations thereof, or the amphiphilic spacer is an alkylthiol or an alkylamine.

28. (canceled)

29. The method of claim 26, wherein the amphiphilic spacer is 1-octadecanethiol.

30. The method of claim 20, wherein the nanosphere further comprises a polymer.

31. The method of claim 30, wherein the polymer is poly(lactide-co-glycolide) (PLGA).

32. The method of claim 20, wherein the nanosphere further comprises a therapeutic agent.

33. (canceled)

34. The method of claim 32, wherein the therapeutic agent is conjugated to a hydrophilic spacer, a hydrophobic spacer, an amphiphilic spacer, or an amphiphilic polymer.
